(19) United States
(12) Patent Application Publication
(10) Pub. No.: US 2024/0279145 A1
(43) Pub. Date: Aug. 22, 2024

(54) PROCESS FOR WORKUP OF A METHANOL/WATER MIXTURE IN THE PRODUCTION OF ALKALI METAL METHOXIDES IN A REACTION COLUMN

(71) Applicant: EVONIK OPERATIONS GMBH, Essen (DE)

(72) Inventors: Dirk ROETTGER, Köln (DE); Moritz SCHRÖDER, Münster (DE); Niklas PAUL, Marl (DE); Armin Matthias RIX, Marl (DE); Philip ZITZEWITZ, Haltern am See (DE); Martin OLDEMEYER, Haltern am See (DE); Martin WÜLLER, Marl (DE); Tanita Valèrie SIX, Dortmund (DE); Johannes RUWWE, Hanau (DE)

(73) Assignee: EVONIK OPERATIONS GMBH, Essen (DE)

(21) Appl. No.: 18/570,519

(22) PCT Filed: Mar. 23, 2022
(86) PCT No.: PCT/EP2022/057591

§ 371 Date: Dec. 14, 2023

(30) Foreign Application Priority Data
Jun. 16, 2021 (EP) 21179722.0

(51) Int. Cl. C07C 29/70; B01D 3/00; B01D 3/14; C07C 29/80; C07C 31/04; C07C 31/30

(52) U.S. Cl. CPC C07C 29/705; B01D 3/007; B01D 3/009; B01D 3/143; C07C 29/80; C07C 31/04; C07C 31/30

(57) ABSTRACT

The present invention relates to a process for workup of a methanol/water mixture which is employed in the production of alkali metal methoxides in a reaction column. The mixture is distillatively separated in a rectification column. The vapours obtained at the upper end of the rectification column are compressed in at least two stages and the energy of the vapours compressed in each case is advantageously transferred to bottoms and side streams of the rectification column. This allows particularly energy-efficient use of the energy of the compressed vapours in the process according to the invention.

The process for workup of a methanol/water mixture is employed in the production of alkali metal methoxides in a reaction column, wherein methanol and alkali metal hydroxide solution are reacted with one another in countercurrent in a reaction column. Alkali metal methoxide dissolved in methanol is withdrawn at the lower end and a methanol/water mixture which is worked up with the workup process according to the invention is withdrawn at the upper end. The energy of the compressed vapours may additionally be used for operating the reaction column or for operating a reaction column in which a process for transalcoholization of alkali metal alkoxides is performed.

The present invention relates to a process for workup of a methanol/water mixture which is employed in the production of alkali metal methoxides in a reaction column. The mixture is distillatively separated in a rectification column. The vapours obtained at the upper end of the rectification column are compressed in at least two stages and the energy of the vapours compressed in each case is advantageously transferred to bottoms and side streams of the rectification column. This allows particularly energy-efficient use of the energy of the compressed vapours in the process according to the invention.

The process for workup of a methanol/water mixture is employed in the production of alkali metal methoxides in a reaction column, wherein methanol and alkali metal hydroxide solution are reacted with one another in countercurrent in a reaction column. Alkali metal methoxide dissolved in methanol is withdrawn at the lower end and a methanol/water mixture which is worked up with the workup process according to the invention is withdrawn at the upper end. The energy of the compressed vapours may additionally be used for operating the reaction column or for operating a reaction column in which a process for transalcoholization of alkali metal alkoxides is performed.

### 1. BACKGROUND OF THE INVENTION

Alcohol/water mixtures are obtained for example in the production of alkali metal alkoxides from aqueous, optionally alcohol-containing, alkali metal hydroxide solution and alcohol. This process employs in particular methanol and ethanol as the alcohol.

Alkali metal alkoxides are used as strong bases in the synthesis of numerous chemicals, for example in the production of pharmaceutical or agrochemical active ingredients. Alkali metal alkoxides are also used as catalysts in transesterification and amidation reactions.

Alkali metal alkoxides (MOR) are produced by reactive distillation of alkali metal hydroxides (MOH) and alcohols (ROH) in a countercurrent distillation column, wherein the water of reaction formed according to the following reaction <1> is removed with the distillate.

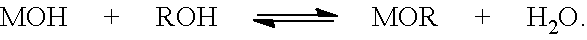

Such a process principle is described, for example, in U.S. Pat. No. 2,877,274 A, wherein aqueous alkali metal hydroxide solution and gaseous methanol are run in countercurrent in a reactive rectification column. This process is described again in generally unchanged form in WO 01/42178 A1.

Similar processes, which, however, additionally employ an entraining agent such as benzene, are described in GB 377,631 A and U.S. Pat. No. 1,910,331 A. This entraining agent is used to separate water and the water-soluble alcohol. In both patent specifications the condensate is subjected to a phase separation to separate off the water of reaction. A further similar process is the reaction of an alkali metal alkoxide with another alcohol in a reaction column (“transalcoholization”) according to DE 27 26 491 A1.

Correspondingly, DE 96 89 03 C describes a process for continuous production of alkali metal alkoxides in a reaction column, wherein the water-alcohol mixture withdrawn at the top of the column is condensed and then subjected to a phase separation. The aqueous phase is discarded and the alcoholic phase is returned to the top of the column together with the fresh alcohol. EP 0 299 577 A2 describes a similar process, wherein the water in the condensate is separated off with the aid of a membrane.

The most industrially important alkali metal alkoxides are those of sodium and potassium, especially the methoxides and ethoxides. Their synthesis is frequently described in the prior art, for example in EP 1 997 794 A1.

The syntheses of alkali metal alkoxides by reactive rectification described in the prior art typically afford vapours comprising the employed alcohol and water. It is advantageous for economic reasons to reuse the alcohol comprised in the vapours as a reactant in the reactive distillation. The vapours are therefore typically supplied to a rectification column and the alcohol present therein is separated off (described for example in GB 737 453 A and U.S. Pat. No. 4,566,947 A). The thus recovered alcohol is then supplied to the reactive distillation as a reactant for example.

Alternatively or in addition a portion of the alcohol vapour may be utilized for heating the rectification column (described in WO 2010/097318 A1). However, this requires that the vapour be compressed in order to achieve the temperature level required for heating the rectification column. Especially a multistage compression of the vapour is thermodynamically advantageous. The vapour is cooled between the compression stages here. The intermediate cooling also ensures that the maximum allowable temperature of the compressor is not exceeded. The disadvantage of this cooling performed in the customary processes is that the energy thus withdrawn dissipates without being utilized.

There is therefore a need for improved processes for workup of an alcohol/water mixture as employable in particular in the context of a process for producing alkali metal alkoxides. This process shall feature a particularly efficient utilization of the energy present in the compressed vapours for operation of the rectification column. The process shall accordingly allow energy-efficient utilization of the heat liberated during compression and cooling of the vapours.

### 2. BRIEF SUMMARY OF THE INVENTION

The present invention relates to a process for producing at least one alkali metal alkoxide of formula MAOR, wherein R is methyl, and wherein M_{A} is a metal selected from sodium, potassium, preferably sodium.

In the process according to the invention for producing at least one alkali metal alkoxide a mixture G comprising water and methanol ROH is obtained which is employed in a process for workup of a mixture G comprising water and alcohol ROH, wherein R is methyl, and is worked up using the process for workup of a mixture G. The process for workup of a mixture G is performed in a rectification column and is accordingly a distillative process.

In a further, preferred aspect the present invention relates to a process for transalcoholization of alkali metal alkoxides. In this process the alcohol of an alkali metal alkoxide M_{c}OR′ is substituted by another alcohol R″OH by reacting M_{c}OR′ with R″OH in a reaction column to afford M_{c}OR″, wherein the process utilizes energy from certain vapour streams from the process according to the invention for workup of a mixture G for operation.

### 3. FIGURES

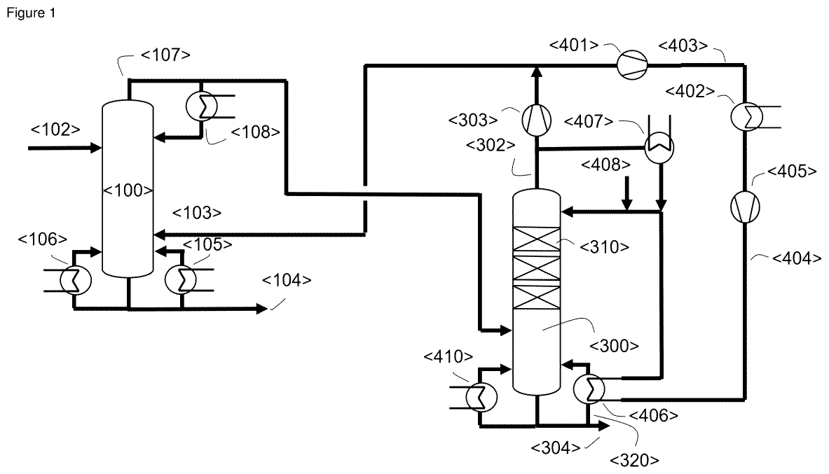

### 3.1 FIG. 1

FIG. 1 shows a noninventive embodiment of a process for producing alkali metal methoxides in which the distillative separation of the methanol-water mixture is carried out as in the prior art (WO 2010/097318 A1, FIG. 1).

Aqueous NaOH S_{AE2} <102> is reacted in a reaction column RR_{A} <100> with methanol S_{AE1} <103> to afford the corresponding sodium alkoxide. At the top of the reaction column RR_{A} <100> an aqueous NaOH solution is added as reactant stream S_{AE2} <102>. It is alternatively also possible to add a methanolic NaOH solution as reactant stream S_{AE2} <102>. To produce the corresponding potassium methoxide aqueous or methanolic KOH solution is added as reactant stream S_{AE2} <102>. Above the bottom of the reaction column RR_{A} <100> methanol is added in vapour form as reactant stream S_{AE1} <103>.

At the bottom of the reaction column RR_{A} <100> a mixture of the corresponding methoxide in the methanol S_{AP*} <104> is withdrawn. The bottoms evaporator V_{SA} <105> and the optional evaporator V_{SA′}<106> at the bottom of the column RR_{A} <100> are used to adjust the concentration of the sodium methoxide solution S_{AP*} <104> to the desired value.

At the top of the reaction column RR_{A} <100> a vapour stream S_{AB} <107> is withdrawn. A portion of the vapour stream S_{AB} <107> is condensed in the condenser K_{RRA} <108> and applied in liquid form to the top of the reaction column RR_{A} <100> as reflux. However, condenser K_{RRA} <108> and the establishment of the reflux are optional.

The obtained vapour S_{AB} <107> is in whole or in part sent to a rectification column, a water/methanol column, RD_{A} <300> as mixture G. The rectification column RD_{A} <300> contains internals <310>. Therein the mixture G is distillatively separated and the methanol is distillatively recovered as vapour S_{OA} <302> overhead.

A reflux may be established at the rectification column RD_{A} <300>. In this case a portion of the vapour S_{OA} <302> is condensed in a condenser K_{RD} <407> and then recycled into the rectification column RD_{A} <300>. In the embodiments where no reflux is established the remaining portion of S_{OA} <302>/the complete vapour stream S_{OA} <302> is precompressed using compressor VD_{AB2} <303>. A portion of this precompressed vapour is recycled to the reaction column RR_{A} <100> where it is employed as reactant stream S_{AE1} <103>.

The remaining portion of S_{OA} <302> is passed to the compressor VDₓ <401> where it is further compressed to afford vapour stream S_{OA1} <403>, from which energy may be withdrawn in the optional intermediate cooler WT_{X} <402>.

The vapour stream S_{OA1} <403> is once again compressed using compressor VDₓ <405> and the resulting vapour S_{OA2} <404> is then sent to the evaporator V_{SRD} <406> at the bottom of the rectification column RD_{A} <300> for heating, after which fresh methanol <408> is optionally added and it is returned to the rectification column RD_{A} <300> as reflux. If a reflux is established at the rectification column RD_{A} <300>, the stream S_{OA2} <404> may be mixed with the reflux, i.e. the condensate, from K_{RD} <407> before recycling to RD_{A} <300>, and fed into RD_{A} <300> together therewith. Obtained at the bottom of the rectification column RD_{A} <300> is a water stream S_{UA} <304> which may be at least partially (stream S_{UA1} <320>) recycled to the rectification column RD_{A} <300>, wherein said stream may be passed through the evaporator V_{SRD} <406> and/or V_{SRD′} <410>.

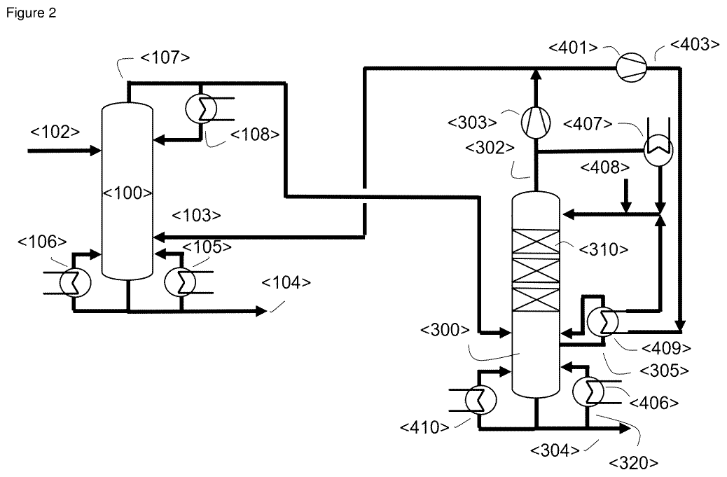

### 3.2 FIG. 2

FIG. 2 shows a further noninventive embodiment of a process which corresponds to the embodiment shown in FIG. 2 of WO 2010/097318 A1.

This embodiment corresponds to the one described in FIG. 1 with the following additional/different features: In addition to the evaporators V_{SRD′} <406> and V_{SRD′} <410> the column bottom of the rectification column RD_{A} <300> comprises an intermediate evaporator V_{ZRD} <409>. A side stream S_{ZA} <305> is withdrawn from the rectification column RD_{A} <300> and passed through V_{ZRD} <409> before being returned to the rectification column RD_{A} <300>. A portion of the vapour stream S_{OA} <302> is precompressed using compressor VD_{AB2} <303> and once a portion thereof has been diverted to the reaction column RR_{A} <100> is compressed in compressor V_{D1} <401> to afford vapour stream S_{OA1} <403> which is sent to the intermediate evaporator V_{ZRD} <409> for heating. Heating in evaporator V_{SRD} <406> or in evaporator V_{SRD′} <410> by S_{OA1} <403> is not carried out. The vapour stream S_{OA} <302> utilized for heating V_{ZRD} <409> is subsequently mixed with the condensate from K_{RD} <407> and the fresh methanol <408> and recycled into the rectification column RD_{A} <300> as reflux. Separate recycling of the reflux from S_{OA} <302> to the rectification column RD_{A} <300> is likewise possible.

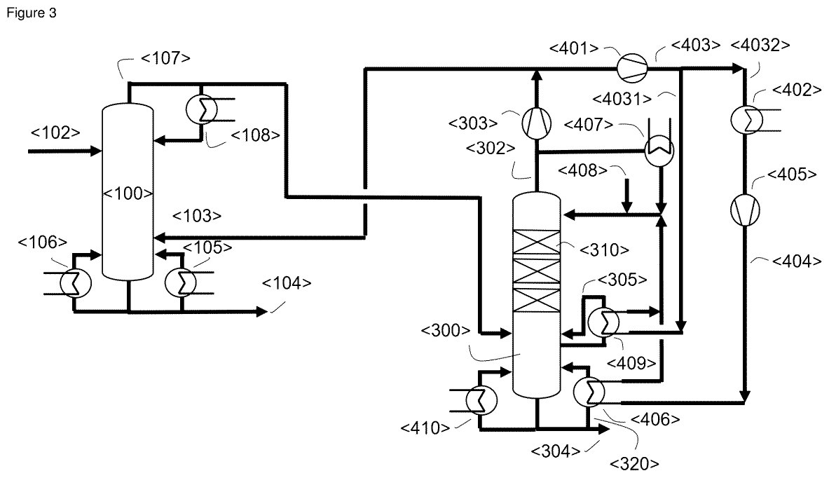

### 3.3 FIG. 3

FIG. 3 shows an embodiment of the process according to the invention. This corresponds to the embodiments described in FIGS. 1 and 2. The rectification column RD_{A} <300> comprises an intermediate evaporator V_{ZRD} <409> and a bottoms evaporator V_{SRD} <406> and optionally the bottoms evaporator V_{SRD′} <410>.

The inventive embodiment has the following differences from the aforementioned embodiments:
1. After compression of a portion of the vapour stream S_{OA} <302> in the compressor V_{D1} <401> the vapour stream S_{OA1} <403> is divided into two portions S_{OA11} <4031> and S_{OA12} <4032>.
2. S_{OA11} <4031> is supplied to the intermediate evaporator V_{ZRD} <409> for heating the stream S_{ZA} <305>.
3. S_{OA12} <4032> is further compressed in the compressor VDₓ <405> to afford stream S_{OA2} <404> and S_{OA2} <404> is sent to the bottoms evaporator V_{SRD} <406> for heating the stream S_{UA1} <320>.
4. Once S_{OA11} <4031> and S_{OA2} <404> have left the respective evaporator V_{ZRD} <409>/V_{SRD} <406> they are combined and the combined stream is mixed with the reflux (i.e. the condensate from K_{RD} <407>) and the fresh methanol <408> and recycled into the rectification column RD_{A} <300>. Separate recycling of these streams to the rectification column RD_{A} <300> is likewise possible.

These differences have the result that the energy of the vapour S_{OA1} <403> may be more efficiently utilized for heating the rectification column RD_{A} <300> compared to the embodiments according to FIGS. 1 and 2.

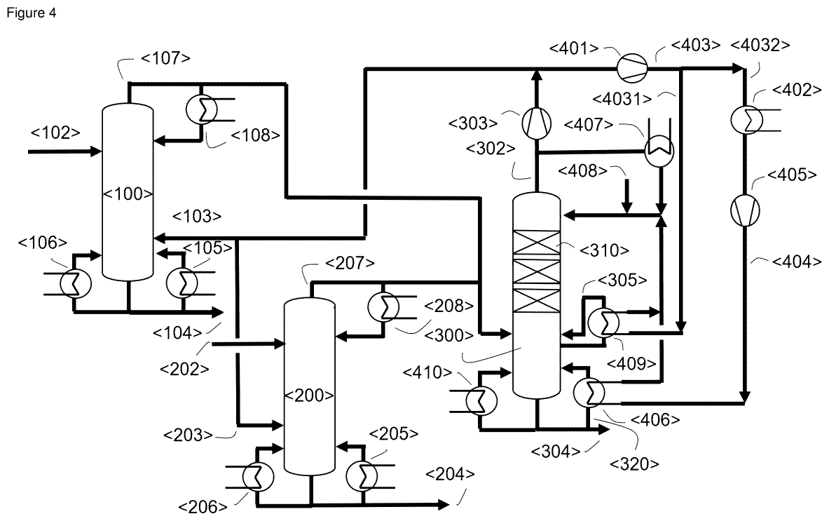

### 3.4 FIG. 4

FIG. 4 shows an embodiment of the process according to the invention. This corresponds to the embodiments described in FIG. 3 with the difference that in a second reaction column RR_{B} <200> an aqueous KOH solution S_{BE2} <202> is reacted with methanol which is also used for reaction in RR_{A} <100>, S_{BE1} <203> to afford the corresponding potassium methoxide.

At the top of the reaction column RR_{B} <200> an aqueous KOH solution is added as reactant stream S_{BE2} <202>. It is alternatively possible to also add a methanolic KOH solution as reactant stream S_{BE2} <202>. Above the bottom of the reaction column RR_{B} <200> methanol is added in vapour form as reactant stream S_{BE1} <203>.

At the bottom of the reaction column RR_{B} <200> a mixture of the corresponding methoxide in methanol S_{BP*} <204> is withdrawn. The bottoms evaporator V_{SB} <205> and the optional evaporator V_{SB′} <206> at the bottom of the column RR_{B} <200> are used to adjust the concentration of the potassium methoxide solution S_{BP*} <204> to the desired value.

At the top of the reaction column RR_{B} <200> a vapour stream S_{BB} <207> is withdrawn. A portion of the vapour stream S_{BB} <207> is condensed in the condenser K_{RRB} <208> and applied in liquid form to the top of the reaction column RR_{B} <200> as reflux. However, condenser K_{RRB} <208> and the establishment of the reflux are optional.

The obtained vapour S_{BB} <207> is supplied to the rectification column RD_{A} <300> in admixture with the portion of the vapour S_{AB} <107> not condensed in the condenser K_{RRA} <108>.

A further difference from the embodiment according to FIG. 3 is that the precompressed portion of the vapour S_{OA} <302> is partially recycled to the reaction column RR_{A} <100> and RR_{B} <200> where it is used as reactant stream S_{AE1} <103>/S_{BE1} <203>.

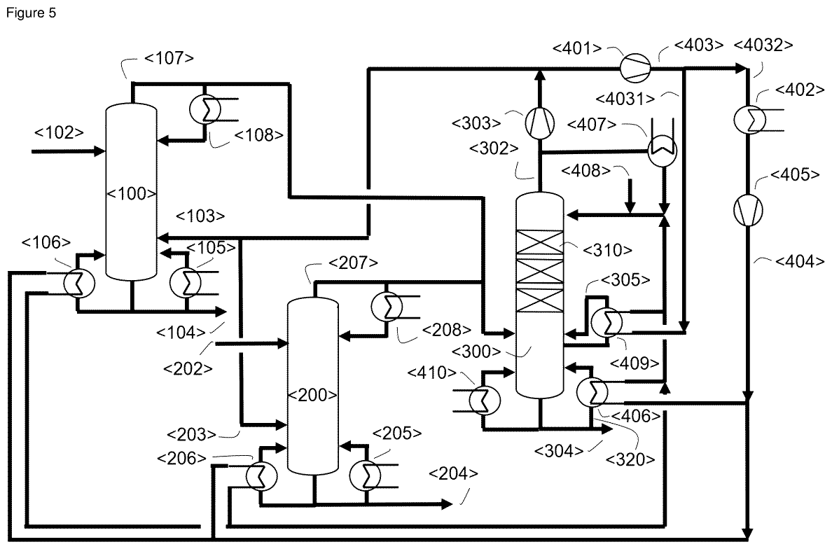

### 3.5 FIG. 5

FIG. 5 shows an embodiment of the process according to the invention. This corresponds to the embodiments described in FIG. 4 with the difference that a portion of S_{OA2} <404> is also utilized for heating the evaporator V_{SA′} <106> at the bottom of the column RR_{A} <100> and the evaporator V_{SB′} <206> at the bottom of the column RR_{B} <200>.

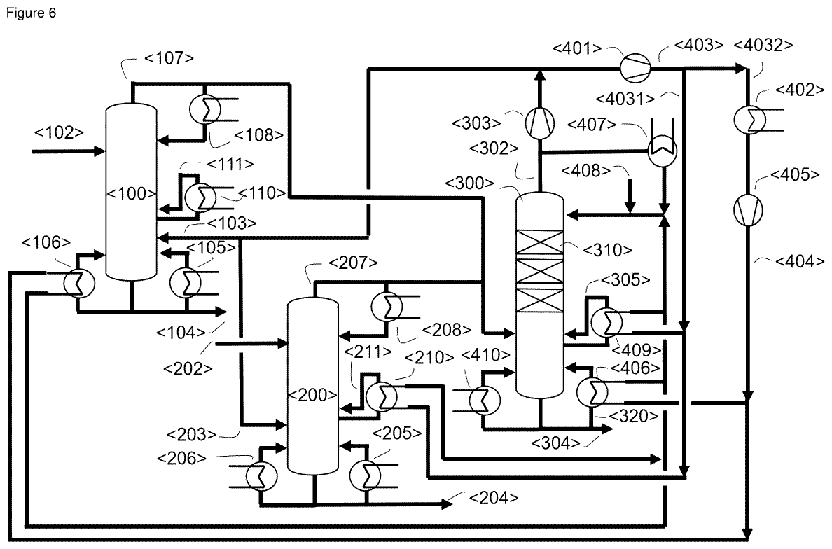

### 3.6 FIG. 6

FIG. 6 shows an embodiment of the process according to the invention. This corresponds to the embodiment described in FIG. 5 with the difference that the reaction columns RR_{A} <100> and RR_{B} <200> each comprise an intermediate evaporator V_{ZA} <110> and V_{ZB} <210>. A side stream S_{ZAA} <111> is withdrawn from the reaction column RR_{A} <100> and passed through V_{ZA} <110> before being returned to the reaction column RR_{A} <100>. A side stream S_{ZBA} <211> is withdrawn from the reaction column RR_{B} <200> and passed through V_{ZB} <210> before being returned to the reaction column RR_{B} <200>.

In a departure from FIG. 5 a portion of S_{OA2} <404> is utilized only for heating the evaporator V_{SA′} <106> at the bottom of the column RR_{A} <100>. A portion of S_{OA11} <4031> is utilized for heating the evaporator V_{ZB} <210> by contrast.

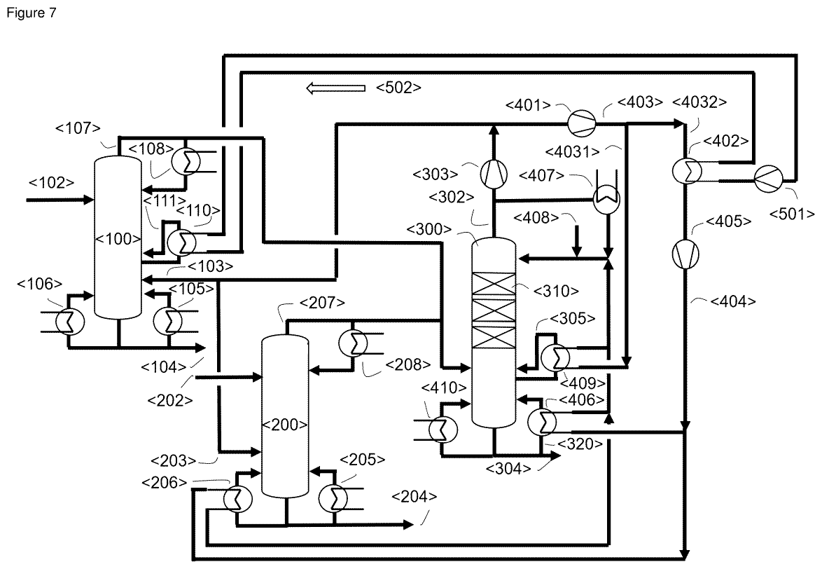

### 3.7 FIG. 7

FIG. 7 shows an embodiment of the process according to the invention. This corresponds to the embodiments described in FIG. 5 with the difference that the reaction column RR_{A} <100> comprises an intermediate evaporator V_{ZA} <110>. A side stream S_{ZAA} <111> is withdrawn from the reaction column RR_{A} <100> and passed through V_{ZA} <110> before being returned to the reaction column RR_{A} <100>. In a departure from FIG. 5 a portion of S_{OA2} <404> is employed only for heating the evaporator V_{SB′} <206> at the bottom of the column RR_{B} <200>. The intermediate evaporator V_{ZA} <110> is heated via a heat transfer medium W <502>, in particular water, transported by a pump <501> which absorbs heat from S_{OA12} <4032> in the intermediate cooler WT_{X} <402> and releases it in the intermediate evaporator V_{ZA} <110>.

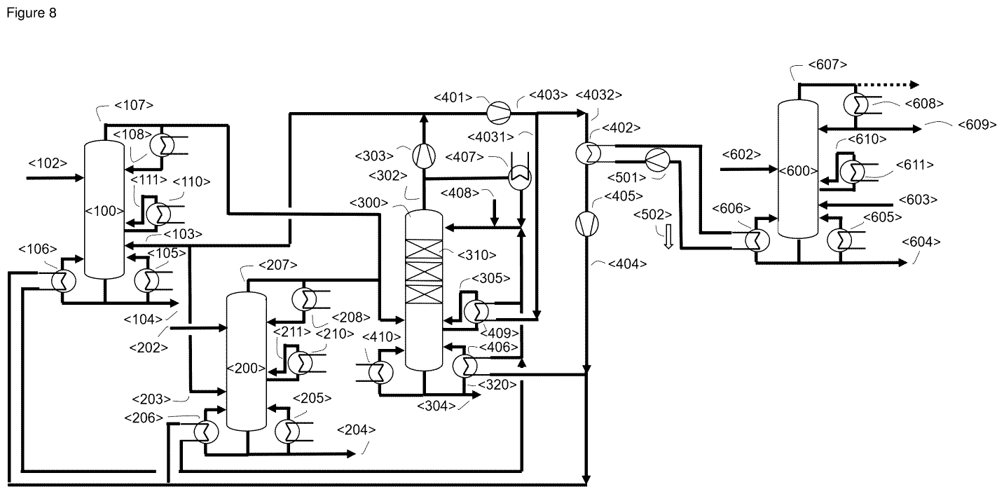

### 3.8 FIG. 8

FIG. 8 shows an embodiment of the process according to the invention. This corresponds to the embodiment described in FIG. 5 with the difference that the reaction columns RR_{A} <100> and RR_{B} <200> each comprise an intermediate evaporator V_{ZA} <110> and V_{ZB} <210>. A side stream S_{ZAA} <111> is withdrawn from the reaction column RR_{A} <100> and passed through V_{ZA} <110> before being returned to the reaction column RR_{A} <100>. A side stream S_{ZBA} <211> is withdrawn from the reaction column RR_{B} <200> and passed through V_{ZB} <210> before being returned to the reaction column RR_{B} <200>.

In addition, FIG. 8 shows a further preferred embodiment of the process according to the invention. It shows a reactive rectification column RR_{C} <600> for transalcoholization of sodium methoxide to sodium ethoxide which is at least partially operated with energy from the stream S_{OA12} <4032>. The column RR_{C} <600> comprises the bottoms evaporators V_{SC} <605> and V_{SC} <606>.

Sodium methoxide solution S_{CE1} <602> is in a reaction column RR_{C} <600> reacted in countercurrent with ethanol S_{CE2} <603> to afford sodium ethoxide and this is withdrawn as an ethanolic solution.

Withdrawn at the bottom of the reaction column RR_{C} <600> is a bottoms product stream S_{CP} <604> comprising sodium ethoxide.

At the top of the reaction column RR_{C} <600> a vapour stream S_{CB} <607> is withdrawn. At least a portion of the vapour stream S_{CB} <607> is condensed in the condenser K_{RRC} <608> and at least a portion thereof is applied in liquid form to the top of the reaction column RR_{C} <600> as reflux. The vapour stream S_{CB} <607> is withdrawn either in gaseous form upstream of the condenser K_{RRC} <608> (marked by dashed line) and/or in liquid form downstream of the condenser K_{RRC} <608> as stream <609>.

A side stream S_{ZC} <610> is preferably withdrawn from the reaction column RR_{C} <600>, wherein energy is transferred to said stream via an intermediate evaporator V_{ZC} <611> and S_{ZC} <610> may subsequently be recycled to RR_{C} <600>.

As the sodium methoxide solution S_{CE1} <602> it is preferable to utilize at least a portion of the bottoms streams S_{AP*} <104> and S_{BP*} <204> obtained in the reaction columns RR_{A} <100> and RR_{B} <200>.

The bottoms evaporator V_{SC′} <606> is heated via a heat transfer medium W₁ <502>, in particular water, transported by a pump <501> which absorbs heat from S_{OA12} <4032> in the intermediate cooler WT_{X} <402> and releases it in the bottoms evaporator V_{SC} <606>.

Alternatively, energy may also be appropriately transferred to the bottoms evaporator V_{SC} <606> or the other bottoms evaporator V_{SC} <605> from another stream selected from S_{OA2} <404>, S_{OA11} <4031> and S_{OA1} <403> before separation into S_{OA11} and S_{OA12}. Energy may likewise be transferred to the ethanol stream S_{CE1} <603>, the sodium methoxide solution S_{CE1} <602> or the side stream S_{ZC} <610> from at least one of the streams S_{OA1} <403>, S_{OA11} <4031>, S_{OA12} <4032>, S_{OA2} <404>.

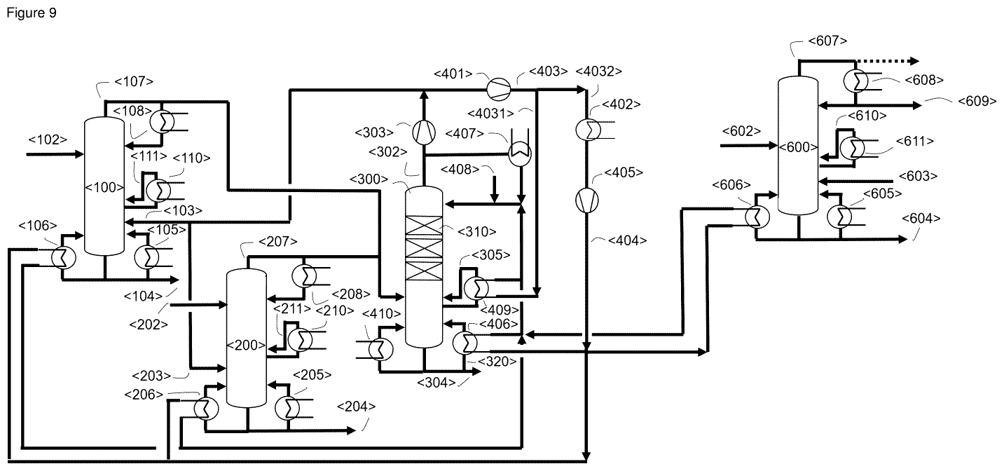

### 3.9 FIG. 9

FIG. 9 shows an embodiment of the process according to the invention. This corresponds to the embodiment described in FIG. 8 with the difference that the heating of the bottoms evaporator V_{SC} <606> is effected directly with a portion of S_{OA}2<404>.

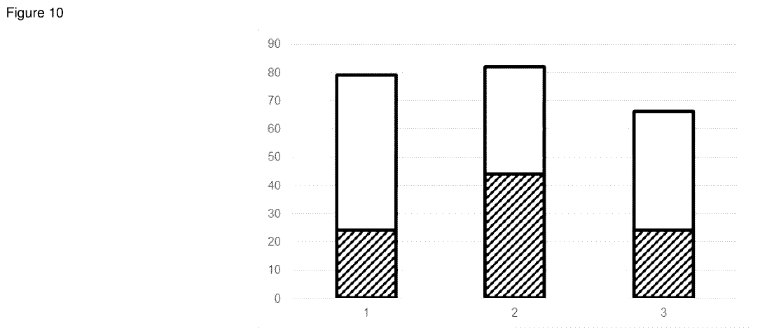

### 3.10 FIG. 10

FIG. 10 illustrates the energy saving in the inventive process according to example 3 compared to the noninventive process according to examples 1 and 2. The x-axis describes the respective example and the y-axis the required power in kilowatts (heating steam and compressor power). The shaded portion of the bars shows the required heating power from low pressure steam while the white portion of the bars shows the sum of the compressor powers.

### 4. DETAILED DESCRIPTION OF THE INVENTION

The present invention relates to a process for producing at least one alkali metal alkoxide of formula MAOR, wherein R is methyl, and wherein M_{A} is a metal selected from sodium, potassium, preferably sodium.

The process according to the invention comprises with steps (a) to (f) a process for workup of a mixture G comprising water and alcohol ROH, wherein R is methyl. ROH is accordingly methanol.

### 4.1 Process for Workup of a Mixture G

The process according to the invention for workup of a mixture G comprises steps (a) to (f) of the process according to the invention for producing at least one alkali metal alkoxide of formula M_{A}OR.

The mixture G is especially gaseous, in which case it is also referred to as “vapours”. The mixture G is the tops stream from a reaction column in which the alcohol ROH has been reacted with an alkali metal hydroxide NaOH or KOH to afford the corresponding alkali metal alkoxide NaOR or KOR.

According to the invention at least a portion of the vapor stream S_{AB} and, if step (α2) is performed, at least a portion of the vapor stream S_{BB} is employed as the mixture G employed in step (a).

The vapor stream S_{AB} is obtained in step (α1) (described at point 4.2.1).

The vapor stream S_{BB} is obtained in optional step (α2) if this step is performed (described at point 4.2.2).

The vapor stream S_{BB} is only obtained if the optional step (α2) is performed. If the optional step (α2) is performed, S_{BB} is obtained and at least a portion of S_{BB} is employed as mixture G, the at least a portion of S_{BB} is employed as mixture G in admixture with S_{AB} or separately from S_{AB}.

In step (β) of the process at least a portion of the vapor stream S_{AB} and, if step (α2) is performed, at least a portion of the vapor stream S_{BB} is employed in admixture with S_{AB} or separately from S_{AB} as mixture G in step (a) of a process for workup of a mixture G comprising water and alcohol ROH.

According to the invention the mixture G comprising water and alcohol ROH is worked up with the process for workup of a mixture G.

### 4.1.1 Step (a) of the Process According to the Invention

In step (a) of the process according to the invention the mixture G is passed into a rectification column RD_{A} and in RD_{A} separated into at least one vapour stream S_{OA} comprising ROH which is withdrawn at the upper end of RD_{A} and at least one stream S_{UA} comprising water which is withdrawn at the lower end of RD_{A}.

“At least one vapour stream S_{OA} comprising ROH which is withdrawn at the upper end of RD_{A}” is to be understood as meaning that the vapour obtained at the upper end of RD_{A} may be withdrawn there as one or more vapour streams. If said stream is withdrawn there in more than one vapour stream, the m vapour streams are referred to as “vapour stream S_{OA1}”, “vapour stream S_{OAIII}”, [ . . . ], “vapour stream S_{OAm}”, wherein “m” indicates the number of vapour streams withdrawn at the upper end of RD_{A} (in Roman numerals).

“At least one vapour stream S_{UA} comprising water which is withdrawn at the lower end of RD_{A}” is to be understood as meaning that water obtained at the lower end of RD_{A} may be withdrawn there as one or more streams. If said stream is withdrawn there in more than one stream, the n streams are referred to as “stream S_{UAI}”, “stream S_{UAII}”, [ . . . ], “stream S_{UAn}”, wherein “n” is the number of streams withdrawn at the lower end of RD_{A} (in Roman numerals).

The mixture G may be introduced to the rectification column RD_{A} via one or more feed points. It is introduced via two or more feed points for example in the embodiments in which step (α2) is performed in the process according to the preferred aspect of the invention and in step (β) at least a portion of the vapour stream S_{BB} is employed separately from S_{AB} as mixture G in step (a) of the process. In this embodiment the mixture G is thus introduced into the rectification column RD_{A} as two separate streams.

In the embodiments of the present invention in which the mixture G is passed into the rectification column RD_{A} as two or more separate streams it is advantageous when the feed points for the individual streams are at substantially the same height on the rectification column RD_{A}.

In a preferred embodiment of step (a) of the process according to the invention the mixture G is in a rectification column RD_{A} separated into a vapour stream S_{OA} comprising ROH which is withdrawn at the upper end of RD_{A} and a stream S_{UA} comprising water which is withdrawn at the lower end of RD_{A}.

Another term for “upper end of a rectification column” is “head”.

Another term for “lower end of a rectification column” is “bottom” or “foot”.

The pressure of the at least one vapour stream S_{OA} is referred to as “p_{OA}” and its temperature as “T_{OA}”. This relates especially to the pressure and temperature of the at least one vapour stream S_{OA} when it is withdrawn from the rectification column RD_{A} in step (a).

The pressure p_{OA} is especially in the range from 0.5 bar abs. to 8 bar abs., more preferably in the range from 0.6 bar abs. to 7 bar abs., more preferably in the range from 0.7 bar abs. to 6 bar abs., yet more preferably in the range from 1 bar abs. to 5 bar abs., and is most preferably 1 bar abs. to 4 bar abs.

The temperature T_{OA} is especially in the range from 45° C. to 150° C., more preferably in the range from 48° C. to 140° C., more preferably in the range from 50° C. to 130° C., yet more preferably in the range from 60° ° C. to 120° C., most preferably in the range from 60° C. to 110° C.

Any desired rectification column known to those skilled in the art may be employed as rectification column RD_{A} in step (a) of the process. The rectification column RD_{A} preferably contains internals. Suitable internals are, for example, trays, unstructured packings or structured packings. As trays, use is normally made of bubble cap trays, sieve trays, valve trays, tunnel trays or slit trays. Unstructured packings are generally beds of random packing elements. Packing elements normally used are Raschig rings, Pall rings, Berl saddles or Intalox® saddles. Structured packings are for example marketed under the trade name Mellapack® from Sulzer. Apart from the internals mentioned, further suitable internals are known to a person skilled in the art and can likewise be used.

Preferred internals have a low specific pressure drop per theoretical plate. Structured packings and random packing elements have, for example, a significantly lower pressure drop per theoretical plate than trays. This has the advantage that the pressure drop in the rectification column RD_{A} remains as low as possible and the mechanical power of the compressor and the temperature of the alkohol/water mixture to be evaporated therefore remain low.

When the rectification column RD_{A} contains structured packings or unstructured packings these may be divided or in the form of an uninterrupted packing. However, at least two packings are typically provided, one packing above the feed point for the mixture G and one packing below the feed point for the mixture G. It is also possible to provide one packing above the feed point for the mixture G and two or more trays below the feed point of the mixture G. If an unstructured packing is used, for example a random packing, the random packing elements are typically disposed on a suitable support grid (for example sieve tray or mesh tray).

In step (a) of the process according to the invention the at least one vapour stream S_{OA} comprising ROH is then withdrawn at the upper end of the rectification column RD_{A}. The preferred mass fraction of ROH in this vapour stream S_{OA} is ≥99% by weight, more preferably ≥99.6% by weight, yet more preferably ≥99.9% by weight, wherein the remainder is especially water.

Withdrawn at the lower end of RD_{A} is at least one stream S_{UA} comprising water which may preferably comprise <1% by weight, more preferably ≤5000 ppmw, yet more preferably ≤2000 ppmw of alcohol.

The withdrawal of the at least one vapour stream S_{OA} comprising ROH at the top of the rectification column RD_{A} is to be in particular understood as meaning in the context of the present invention that the at least one vapour stream S_{OA} is withdrawn above the internals in the rectification column RD_{A} as a top stream or as a side stream.

The withdrawal of the at least one stream S_{UA} comprising water at the bottom of the rectification column RD_{A} is to be in particular understood as meaning in the context of the present invention that the at least one stream S_{UA} is withdrawn as a bottoms stream or at the lower tray of the rectification column RD_{A}.

The rectification column RD_{A} is operated with or without, preferably with, reflux.

“With reflux” is to be understood as meaning that the vapour stream S_{OA} withdrawn at the upper end of the rectification column RD_{A} is not completely discharged but rather partially condensed and returned to the respective rectification column RD_{A}. In the cases where such a reflux is established the reflux ratio is preferably 0.0001 to 1, more preferably 0.0005 to 0.9, yet more preferably 0.001 to 0.8.

A reflux may be established such that a condenser K_{RD} is attached at the top of the rectification column RD_{A}. In the condenser K_{RD} the respective vapour stream S_{OA} is partially condensed and returned to the rectification column RD_{A}. Generally and in the context of the present invention a reflux ratio is to be understood as meaning the ratio of the proportion of the mass flow withdrawn from the column (kg/h) that is recycled to the respective column in liquid form (reflux) to the proportion of this mass flow (kg/h) that is discharged from the respective column in liquid form or gaseous form.

### 4.1.2 Step (b) of the Process According to the Invention

In step (b) of the process according to the invention at least one side stream S_{ZA} is withdrawn from RD_{A} and recycled to RD_{A}.

In a preferred embodiment of step (b) of the process according to the invention a side stream S_{ZA} is withdrawn from RD_{A} and recycled to RD_{A}.

“Side stream S_{ZA} from RD_{A}” is according to the invention to be understood as meaning that the stream is withdrawn at a withdrawal point E_{ZA} below the head and above the bottom of RD_{A} and in particular additionally recycled to RD_{A} at a feed point Z_{ZA} (this is the point at which the respective side stream S_{ZA} is recycled to the respective rectification column RD_{A}) below the head and above the bottom of RD_{A}.

This is to be understood as meaning in particular that the withdrawal point E_{ZA} and preferably also the feed point Z_{ZA} of the respective side stream S_{ZA} on the rectification column RD_{A} is below the withdrawal points E_{OA} for all vapour streams S_{OA} withdrawn from RD_{A}, preferably at least 1, more preferably at least 5, yet more preferably at least 10, theoretical plates below the withdrawal point E_{OA} for the vapour stream S_{OA} withdrawn from RD_{A} whose withdrawal point E_{OA} is furthest down the rectification column RD_{A}.

This is also to be understood as meaning in particular that the withdrawal point E_{ZA} and preferably also the feed point Z_{ZA} of the respective side stream S_{ZA} on the rectification column RD_{A} is above the withdrawal points E_{UA} for all streams S_{UA} withdrawn from RD_{A}, preferably at least 1, more preferably at least 2, yet more preferably at least 4, theoretical plates above the withdrawal point E_{UA} for the stream S_{UA} whose withdrawal point E_{UA} is furthest up the rectification column RD_{A}.

In the cases in which at least one vapour stream S_{OA} is at least partially recycled into the rectification column RD_{A} (which is the case when a reflux is established at the rectification column RD_{A} for example) the feed point Z_{OA} (i.e. the point at which the at least one vapour stream S_{OA} is at least partially recycled into the rectification column RD_{A}) of the at least one vapour stream S_{OA} is especially also above the withdrawal points E_{ZA} and especially also above the feed points Z_{ZA} for all side streams S_{ZA} withdrawn from RD_{A}, preferably at least 1, more preferably at least 5, yet more preferably at least 10, theoretical plates above the highest point of all withdrawal and feed points for all side streams S_{ZA} withdrawn from RD_{A}.

In the cases in which at least one stream S_{UA} is at least partially recycled into the rectification column RD_{A} the feed point Z_{UA} (i.e. the point at which the at least one stream S_{UA} is at least partially recycled into the rectification column RD_{A}) of the at least one stream S_{UA} is especially also below the withdrawal points E_{ZA} and especially also below the feed points Z_{ZA} for all side streams S_{ZA} withdrawn from RD_{A}, preferably at least 1, more preferably at least 2, yet more preferably at least 4, theoretical plates below the lowest point of all withdrawal and feed points for all side streams S_{ZA} withdrawn from RD_{A}.

The withdrawal point E_{ZA} of the side stream S_{ZA} and the feed point Z_{ZA} of the side stream S_{ZA} on the rectification column RD_{A} may be positioned between the same trays of RD_{A}. However, they may also be at different heights.

In a preferred embodiment of the process according to the invention the withdrawal point E_{ZA} and preferably also the feed point Z_{ZA} of the at least one side stream S_{ZA} on the rectification column RD_{A} are below the feed point Z_{G}, by which the mixture G is passed into the rectification column RD_{A}, and above the bottom of RD_{A}. It is yet more preferable when the withdrawal point E_{ZA} and preferably also the feed point Z_{ZA} of the at least one side stream S_{ZA} on the rectification column RD_{A} are also below the rectification section of RD_{A}.

In a particularly preferred embodiment of the process according to the invention the withdrawal point E_{ZA} and more preferably also the feed point Z_{ZA} of the at least one side stream S_{ZA} on the rectification column RD_{A} are in the upper ⅘, preferably upper ¾, preferably upper 7/10, more preferably upper ⅔, more preferably upper ½, of the region of the rectification column RD_{A} below the feed point Z_{G}, by which the mixture G is passed into the rectification column RD_{A}, and above the uppermost of all withdrawal and feed points for all streams S_{UA} withdrawn from RD_{A}. It is yet more preferable when the withdrawal point E_{ZA} and preferably also the feed point Z_{ZA} of the at least one side stream S_{ZA} on the rectification column RD_{A} are then also below the rectification section of RD_{A}.

In a further particularly preferred embodiment of the process according to the invention the rectification column RD_{A} contains a rectification section and the withdrawal point E_{ZA} and more preferably also the feed point Z_{ZA} of the at least one side stream S_{ZA} on the rectification column RD_{A} are in the upper ⅘, preferably upper ¾, preferably upper 7/10, more preferably upper ⅔, more preferably upper ½, of the region of the rectification column RD_{A} below the rectification section and above the uppermost of all withdrawal and feed points for all streams S_{UA} withdrawn from RD_{A}.

### 4.1.3 Step (c) of the Process According to the Invention

In step (c) of the process according to the invention at least a portion of the at least one vapour stream S_{OA} (“at least a portion of the at least one vapour stream S_{OA}”=“at least a portion of S_{OA}”) is compressed. This affords a vapour stream S_{OA1} which is compressed with respect to S_{OA}.

The pressure of the vapour stream S_{OA1} is referred to as “p_{OA1}” and its temperature as “T_{OA1}”.

The pressure p_{OA1} is higher than p_{OA}. The precise value of p_{OA1} may be adjusted by those skilled in the art according to the requirements in step (d) provided that the condition p_{OA1}>p_{OA} is met. The quotient of p_{OA1}/p_{OA} (pressures in bar abs. in each case) is preferably in the range from 1.1 to 10, more preferably 1.2 to 8, more preferably 1.25 to 7, most preferably 1.3 to 6.

The temperature T_{OA1} is especially higher than the temperature T_{OA} and the quotient of T_{OA1}/T_{OA} (temperature in ° C. in each case) is preferably in the range from 1.03 to 10, more preferably 1.04 to 9, more preferably 1.05 to 8, more preferably 1.06 to 7, more preferably 1.07 to 6, most preferably 1.08 to 5.

The preferred values of p_{OA1} and T_{OA1} also apply with preference to S_{OA11} and S_{OA12}.

The compressing of the at least a portion of the vapour stream S_{OA} in step (c) may be carried out in any desired manner known to those skilled in the art. The compression may therefore be performed for example mechanically and as a single-stage or multi-stage compression, preferably a multi-stage compression. A multi-stage compression may employ a plurality of compressors of the same type or compressors of different types. A multi-stage compression may be carried out with one or more compressors. The use of single-stage compression or multi-stage compression depends on the compression ratio and thus on the pressure to which the vapour S_{OA} is to be compressed.

Any desired compressor, preferably mechanical compressor, known to those skilled in the art and capable of compressing gas streams is suitable as a compressor in the process according to the invention, in particular for compressing the vapour streams S_{OA} to S_{OA1} or S_{OA12} to S_{OA2}. Suitable compressors are for example single-stage or multi-stage turbines, piston compressors, screw compressors, centrifugal compressors or axial compressors.

In a multi-stage compression compressors suitable for the respective pressure stages to be overcome are employed.

### 4.1.4 Step (d) of the Process According to the Invention

In step (d) of the process according to the invention energy is transferred from a first portion S_{OA11} of the compressed vapour stream S_{OA1} to S_{ZA} before S_{ZA} is recycled to RD_{A}.

S_{OA1} is especially in step (d) initially divided into at least two portions S_{OA11} and S_{OA12}. The ratio of the mass flows (in kg/h) of S_{OA11} to S_{OA12} is preferably in the range from 1:99 to 99:1, more preferably in the range from 1:50 to 50:1, yet more preferably in the range from 1:20 to 30:1, yet more preferably in the range from 5:20 to 15:1.

In step (d) of the process according to the invention energy is transferred from the first portion S_{OA11} to S_{ZA}. Step (d) reduces the energy of S_{OA11} and the stream S_{OA11} thus especially undergoes at least partial condensation.

According to the invention “transfer of energy” is especially to be understood as meaning “heating”, i.e. transfer of energy in the form of heat.

“Transfer of energy from a first portion S_{OA11} of the compressed vapour stream S_{OA1} to S_{ZA}” also comprises the cases in which a portion of S_{OA11} is separated and energy is transferred to S_{ZA} only from this portion. This is the case for example in embodiments of the invention in which energy is additionally transferred from S_{OA11} to the crude product RP_{A} and if step (α2) is performed alternatively or in addition to the crude product RP_{B} (described in section 4.2).

The transfer of energy from S_{OA11} to S_{ZA}, preferably the heating of S_{ZA} by S_{OA11}, is preferably effected directly or indirectly.

“Directly” is to be understood as meaning that S_{OA11} is contacted with S_{ZA} without mixing of the two streams, thus transferring energy, in particular heat, from S_{OA11} to S_{ZA}.

This may be performed by passing S_{OA11} and S_{ZA} through an intermediate evaporator V_{ZRD} on the rectification column RD_{A} so that S_{OA11} effects heating of S_{ZA}.

The employed heat exchangers, in particular the heat exchangers WT_{X}, WT_{Y}, WT_{Z} mentioned hereinbelow, may be heat exchangers known to those skilled in the art, in particular evaporators. In step (d) of the process according to the invention the transfer of energy, preferably heat, from S_{OA11} to S_{ZA} is effected in particular in an intermediate evaporator V_{ZRD}.

“Indirectly” is especially to be understood as meaning that S_{OA11} is contacted with a heat transfer medium W₁, preferably via at least one heat exchanger WT_{X}, wherein the heat transfer medium is not S_{ZA}, and W₁ is thus distinct from S_{ZA}, thus transferring energy, preferably heat, from S_{OA11} to W₁ without the two streams mixing, and the heat is then transferred from W₁ to S_{ZA} by contacting W₁ and S_{ZA}, wherein S_{ZA} and W₁ do or do not mix, but preferably do not mix. If W₁ and S_{ZA} do not mix, the transfer of energy, preferably heat, is effected in particular in a further heat exchanger WT_{Y}.

In a further embodiment of the process according to the invention in the case of indirect energy transfer from S_{OA11} to S_{ZA}, in particular heating of S_{ZA} by S_{OA11}, energy, preferably heat, may also be initially transferred from S_{OA11} to W₁, preferably by contacting via at least one heat exchanger WT_{X}, and then transferred from W₁ to a further heat transfer medium W₂ distinct from S_{ZA}, preferably by contacting via at least one heat exchanger WT_{Y}. The last step then effects transfer of the heat from W₂ to S_{ZA}, wherein S_{ZA} and W₂ do or do not mix, but preferably do not mix. If W₂ and

S_{ZA} do not mix, the transfer of energy, preferably heat, is effected in particular in a further heat exchanger WT_{Z}.

It will be appreciated that still further heat transfer media W₃, W₄, W₅ etc. may accordingly be employed in further embodiments of the present invention.

Utilizable heat transfer media W₁ and further heat transfer media W₂, W₃, W₄, W₅ include any heat transfer media known to those skilled in the art, preferably selected from the group consisting of water; alcohol-water solutions; salt-water solutions, also including ionic liquids such as for example LiBr solutions, dialkylimidazolium salts such as especially dialkylimidazolium dialkylphosphates; mineral oils, for example diesel oils; thermal oils such as for example silicone oils; biological oils such as for example limonene; aromatic hydrocarbons such as for example dibenzyltoluene. The most preferred heat transfer medium W₁ is water.

Salt-water solutions that may be used are also described for example in DE 10 2005 028 451 A1 and WO 2006/134015 A1.

After step (d) S_{OA11} may then be returned to rectification column RD_{A}, optionally together with fresh alcohol and/or with the reflux for rectification column RD_{A}. In a preferred embodiment further energy is transferred from S_{OA11}, especially after the transfer of energy to S_{ZA}.

In a preferred embodiment of the process according to the invention once S_{OA11} has transferred energy to S_{ZA} according to step (d) energy, preferably heat, is transferred from S_{OA11} to S_{OA}, in particular to the portion of S_{OA} sent to a compression, preferably the compression in step (c), wherein this may be a precompression of S_{OA} or the compression of S_{OA} to S_{OA1}. This is preferably the first compression to which the stream S_{OA} is subjected once it has left the column RD_{A}. This makes it possible to employ a portion of the residual energy/residual heat still stored by S_{OA11} in the process, in this case for heating of S_{OA} to be compressed. This evaporates any droplets present in S_{OA} and thus prevents entry of droplets into the compressor.

Other, preferred additional sinks for the energy, preferably heat, in S_{OA11} are described hereinbelow (see paragraph 4.3).

Step (d) of the process according to the invention reflects one aspect of the unexpected effect of the present invention. The excess energy obtained upon compression of the vapour stream S_{OA} to afford the compressed vapour stream S_{OA1} does not dissipate without being utilized but rather is employed in the rectification. This is effected such that S_{OA} is initially compressed to afford S_{OA1}, thus allowing adjustment to a value optimal for energy transfer from S_{OA11} to S_{ZA}, and then a portion S_{OA12} that is distinct from S_{OA11} may be further compressed to afford S_{OA2}. The heat of condensation obtained upon further compression of S_{OA12} to afford S_{OA2} is introduced into the column in the bottoms evaporator. The required additional compressor power is less than the heating steam power saved thereby. The process according to the invention requires less energy than those of the prior art, such as are shown in examples 1 and 2. The compression to afford S_{OA2} allows the pressure and temperature of S_{OA2} to be adjusted such that optimal energy transfer from S_{OA2} to S_{UA1}/S_{UA} may be effected.

### 4.1.5 Step (e) of the Process According to the Invention

In step (e) of the process of the invention a portion S_{OA12} of the compressed vapour stream S_{OA1} that is distinct from S_{OA11} is subjected to further compression to afford a vapour stream S_{OA2} that is compressed relative to S_{OA11}.

It will be appreciated that after performance of step (e) S_{OA2} is also compressed relative to S_{OA12} and S_{OA1}.

The pressure of the vapour stream S_{OA2} is referred to as “p_{OA2}” and its temperature as “T_{OA2}”.

The pressure p_{OA2} is higher than p_{OA} and the quotient of p_{OA2}/p_{OA1} (pressures in bar abs. in each case) is preferably in the range from 1.1 to 10, more preferably 1.2 to 8, more preferably 1.25 to 7, most preferably 1.3 to 6.

The temperature T_{OA2} is especially higher than the temperature T_{OA1} and the quotient of T_{OA2}/T_{OA1} (temperature in ° C. in each case) is preferably in the range from 1.03 to 10, more preferably 1.04 to 9, more preferably 1.05 to 8, more preferably 1.06 to 7, more preferably 1.07 to 6, most preferably 1.08 to 5.

The compressing of S_{OA12} in step (e) may be performed by processes known to those skilled in the art. The compression may therefore be performed for example mechanically and as a single-stage or multi-stage compression, preferably a multi-stage compression. A multi-stage compression may employ a plurality of compressors of the same type or compressors of different types. The use of single-stage compression or multi-stage compression depends on the pressure to which the vapour S_{OA12} is to be compressed. A precompression described for S_{OA} in the context of step (c) may also be performed for the compression of S_{OA12} to S_{OA2} but compression in one stage, i.e. using a compressor VDₓ, is especially sufficient in step (e).

### 4.1.6 Step (f) of the Process According to the Invention

In step (f) of the process according to the invention energy is transferred from at least a portion of S_{OA2} to at least a portion S_{UA1} of the at least one stream S_{UA} before S_{UA1} is recycled to RD_{A}.

In step (f) of the process energy is preferably transferred from at least a portion of S_{OA2} to a portion S_{UA1} of the at least one stream S_{UA} before S_{UA1} is recycled to RD_{A}.

Step (f) reduces the energy of S_{OA2} and the stream S_{OA2} thus especially undergoes at least partial condensation.

Step (f) of the process according to the invention comprises the following preferred embodiments (f1), (f2), (f3):
(f1) energy is transferred from at least a portion of S_{OA2} to a portion S_{UA1} of the at least one stream S_{UA} and S_{UA1} is then recycled to RD_{A};
(f2) energy is transferred from at least a portion of S_{OA2} to a portion S_{UA1′} of the at least one stream S_{UA} and a portion S_{UA1} of S_{UA1′} is then recycled to RD_{A};
(f3) energy is transferred from at least a portion of S_{OA2} to the whole of stream S_{UA} and then the whole of stream S_{UA} or only a portion S_{UA1} of stream S_{UA}, preferably only a portion S_{UA1} of stream S_{UA}, is recycled into RD_{A}.

The transfer of energy from at least a portion of S_{OA2} to the at least a portion S_{UA1} of the at least one stream S_{UA}, preferably the heating of the at least a portion S_{UA1} of the at least one stream S_{UA} by at least a portion of S_{OA2}, is preferably effected directly or indirectly.

“Directly” is to be understood as meaning that at least a portion of S_{OA2} is contacted with the at least a portion S_{UA1} of the at least one stream S_{UA} without mixing of the two streams, thus transferring energy, in particular heat, from at least a portion of S_{OA2} to the at least a portion S_{UA1} of the at least one stream S_{UA}.

This may be performed by passing the at least a portion of S_{OA2} and the at least a portion S_{UA1} of the at least one stream S_{UA} through an bottoms evaporator V_{SRD} on the rectification column RD_{A} SO that S_{OA11} effects heating of the at least a portion S_{UA} of the at least one stream S_{UA}.

The employed heat exchangers, in particular the heat exchangers WT_{X}, WT_{Y}, WT_{Z} mentioned hereinbelow, may be heat exchangers known to those skilled in the art, in particular evaporators. In step (f) of the process according to the invention the transfer of energy, preferably heat, from at least a portion of S_{OA2} to the at least a portion S_{UA1} of the at least one stream S_{UA} is in particular effected in a bottoms evaporator V_{SRD}.

“Indirectly” is especially to be understood as meaning that the at least a portion of S_{OA2} is contacted with at least one heat transfer medium W₁ preferably via at least one heat exchanger WT_{X}, wherein the heat transfer medium is not the at least a portion S_{UA} of the at least one stream S_{UA} and W₁ is thus distinct therefrom, thus transferring energy, preferably heat, from the at least a portion of S_{OA2} to the at least one heat transfer medium W₁ without the two streams mixing, and the heat is then transferred from W₁ to the at least a portion S_{UA} of the at least one stream S_{UA} by contacting W₁ with the relevant component, wherein the at least a portion S_{UA1} of the at least one stream S_{UA} and W₁ do or do not mix, but preferably do not mix.

In a further embodiment of the process according to the invention in the case of indirect energy transfer from at least a portion of S_{OA2} to the at least a portion S_{UA1} of the at least one stream S_{UA}, in particular heating of the at least a portion S_{UA1} of the at least one stream S_{UA} by the at least a portion of S_{OA2}, energy, preferably heat, may also be initially transferred from S_{OA2} to W₁, preferably by contacting via at least one heat exchanger WT_{X}, and then transferred from W₁ to a further heat transfer medium W₂ distinct from the at least a portion S_{UA1} of the at least one stream S_{UA}, preferably by contacting via at least one heat exchanger WT_{Y}. The last step then effects transfer of the heat from W₂ to the at least a portion S_{UA} of the at least one stream S_{UA}, wherein the at least a portion S_{UA1} of the at least one stream S_{UA} and W₂ do or do not mix, but preferably do not mix. It will be appreciated that still further heat transfer media W₃, W₄, W₅ etc. may accordingly be employed in further embodiments of the present invention.

Utilizable heat transfer media W₁ and further heat transfer media W₂, W₃, W₄, W₅ used include any heat transfer media known to those skilled in the art, preferably selected from the group consisting of water; alcohol-water solutions; salt-water solutions, also including ionic liquids such as for example LiBr solutions, dialkylimidazolium salts such as especially dialkylimidazolium dialkylphosphates; mineral oils, for example diesel oils; thermal oils such as for example silicone oils; biological oils such as for example limonene; aromatic hydrocarbons such as for example dibenzyltoluene. The most preferred heat transfer medium W₁ is water.

Salt-water solutions that may be used are also described for example in DE 10 2005 028 451 A1 and WO 2006/134015 A1.

After step (f) the at least a portion of S_{OA2} may then be returned to the rectification column RD_{A}, optionally together with fresh alcohol and/or with the reflux for the rectification column RD_{A} and/or with the stream S_{OA11} obtained after performance of step (d). In a preferred embodiment further energy is transferred from at least a portion of S_{OA2}, especially after the transfer of energy to the at least a portion S_{UA1} of S_{UA}.

In a preferred embodiment of the process according to the invention once energy has been transferred from the at least a portion of S_{OA2} to the at least a portion S_{UA1} of S_{UA} according to step (f) energy, more preferably heat, is transferred from the at least a portion of S_{OA2} to S_{OA}, in particular to the portion of S_{OA} which is sent to a compression, preferably the compression in step (c), wherein this may be a precompression of S_{OA} or the compression of S_{OA} to afford S_{OA1}. This is preferably the first compression to which the stream S_{OA} is subjected once it has left the column RD_{A}. This makes it possible to employ a portion of the residual energy/residual heat still stored by the at least a portion of S_{OA2} in the process, in this case for heating of S_{OA} to be compressed.

Other, preferred additional sinks for the energy, preferably heat, in the at least a portion of S_{OA2} are described hereinbelow (see paragraph 4.3).

### 4.2 Process for Producing at Least One Alkali Metal Alkoxide

The mixture G employed in the process for workup of a mixture G according to the invention is a water/methanol mixture withdrawn from a reaction column for producing alkali metal alkoxides.

The present invention thus relates to a process for producing at least one alkali metal alkoxide of formula M_{A}OR, wherein R is methyl, and wherein M_{A} is a metal selected from sodium, potassium, preferably sodium.

ROH is accordingly methanol.

### 4.2.1 Step (α1)

In step (α1) of the process according to the invention a reactant stream S_{AE1} comprising ROH is reacted with a reactant stream S_{AE2} comprising M_{A}OH in countercurrent in a reactive rectification column RR_{A} to afford a crude product RP_{A} comprising M_{A}OR, water, ROH, M_{A}OH.

According to the invention, a “reactive rectification column” is a rectification column in which the reaction according to step (α1) or step (α2) of the process of the invention proceeds at least in some parts is defined. It may also be abbreviated to “reaction column”.

In step (α1) a bottoms product stream S_{AP} comprising ROH and M_{A}OR is withdrawn at the lower end of RR_{A}. A vapour stream S_{AB} comprising water and ROH is withdrawn at the upper end of RR_{A}.

M_{A} is selected from sodium, potassium, preferably sodium.

The reactant stream S_{AE1} comprises ROH. In a preferred embodiment the mass fraction of ROH in S_{AE1} is ≥95% by weight, yet more preferably ≥99% by weight, wherein S_{AE1} otherwise comprises especially water.

The alcohol ROH employed in step (α1) as reactant stream S_{AE1} may also be commercially available alcohol having an alcohol mass fraction of more than 99.8% by weight and a mass fraction of water of up to 0.2% by weight.

The reactant stream S_{AE1} is preferably introduced in vapour form.

The reactant stream S_{AE2} comprises M_{A}OH. In a preferred embodiment S_{AE2} comprises not only M_{A}OH but also at least one further compound selected from water, ROH. It is yet more preferable when S_{AE2} comprises water in addition to M_{A}OH, thus rendering S_{AE2} an aqueous solution of M_{A}OH.

When the reactant stream S_{AE2} comprises M_{A}OH and water the mass fraction of M_{A}OH based on the total weight of the aqueous solution forming S_{AE2} is especially in the range from 10% to 75% by weight, preferably from 15% to 54% by weight, more preferably from 30% to 53% by weight and particularly preferably from 40% to 52% by weight.

When the reactant stream S_{AE2} comprises M_{A}OH and ROH the mass fraction of M_{A}OH in ROH based on the total weight of the solution forming S_{AE2} is especially in the range from 10% to 75% by weight, preferably from 15% to 54% by weight, more preferably from 30% to 53% by weight and particularly preferably from 40% to 52% by weight.

In the particular case in which the reactant stream S_{AE2} comprises both water and ROH in addition to M_{A}OH it is particularly preferable when the mass fraction of M_{A}OH in ROH and water based on the total weight of the solution forming S_{AE2} is especially in the range from 10% to 575% by weight, preferably from 15% to 54% by weight, more preferably from 30% to 53% by weight and particularly preferably from 40% to 52% by weight.

Step (α1) is performed in a reactive rectification column (or “reaction column”) RR_{A}.

Step (α2), explained further hereinbelow, is performed in a reactive rectification column (or “reaction column”) RR_{B}.

The reaction column RR_{A}/RR_{B} preferably contains internals. Suitable internals are, for example, trays, structured packings or unstructured packings. When the reaction column RR_{A}/RR_{B} contains trays, then bubble cap trays, valve trays, tunnel trays, Thormann trays, cross-slit bubble cap trays or sieve trays are suitable. When the reaction column RR_{A}/RR_{B} contains trays it is preferable to choose trays where not more than 5% by weight, more preferably less than 1% by weight, of the liquid trickles through the respective trays. The constructional measures required to minimize trickle-through of the liquid are familiar to those skilled in the art. In the case of valve trays, particularly tightly closing valve designs are selected for example. Reducing the number of valves also makes it possible to increase the vapour velocity in the tray openings to twice the value typically established. When using sieve trays it is particularly advantageous to reduce the diameter of the tray openings while maintaining or even increasing the number of openings.

When using structured or unstructured packings, structured packings are preferred in terms of uniform distribution of the liquid.

For columns comprising unstructured packings, especially comprising random packings, and for columns comprising structured packings, the desired characteristics of the liquid distribution may be achieved when the liquid trickling density in the edge region of the column cross section adjacent to the column shell, corresponding to about 2% to 5% of the total column cross section, is reduced compared to the other cross-sectional regions by up to 100%, preferably by 5% to 15%. This can easily be achieved by, for example, targeted distributions of the drip points of the liquid distributors or the holes thereof.

The process according to the invention may be performed either continuously or discontinuously. It is preferably performed continuously.

“Reaction of a reactant stream S_{AE1} comprising ROH with a reactant stream S_{AE2} comprising M_{A}OH in countercurrent” is according to the invention achieved, in particular, as a result of the feed point for at least a portion of the reactant stream S_{AE1} comprising ROH in step (α1) being located on the reaction column RR_{A} below the feed point of the reactant stream S_{AE2} comprising M_{A}OH.

The reaction column RR_{A} preferably comprises at least 2, in particular 15 to 40, theoretical trays between the feed point of the reactant stream S_{AE1} and the feed point of the reactant stream S_{AE2}.

The reaction column RR_{A} may be operated as a pure stripping column. In this case the reactant stream S_{AE1} comprising ROH is introduced in vapour form in the lower region of the reaction column RR_{A}.

Step (α1) also encompasses the case where a portion of the reactant stream S_{AE1} comprising ROH is added in vapour form below the feed point of the reactant stream S_{AE2} comprising aqueous sodium hydroxide solution M_{A}OH but nevertheless at the upper end or in the region of the upper end of the reaction column RR_{A}. This makes it possible to reduce the dimensions of the lower region of the reaction column RR_{A}. When a portion of the reactant stream S_{AE1} comprising ROH, in particular methanol, is added especially in vapour form at the upper end or in the region of the upper end of the reaction column RR_{A} only a fraction of 10% to 70% by weight, preferably of 30% to 50% by weight, (in each case based on the total amount of the alcohol ROH employed in step (α1)) is introduced at the lower end of the reaction column RR_{A} and the remaining fraction is added in vapour form in a single stream or divided into a plurality of substreams, preferably 1 to 10 theoretical trays, particularly preferably 1 to 3 theoretical trays, below the feed point of the reactant stream S_{AE2} comprising M_{A}OH.

In the reaction column RR_{A}, the reactant stream S_{AE1} comprising ROH is then reacted with the reactant stream S_{AE2} comprising M_{A}OH according to the reaction <1> described hereinabove to afford M_{A}OR and H₂O, where these products are present in admixture with the reactants ROH and M_{A}OH since an equilibrium reaction is concerned. Accordingly, a crude product RP_{A} which comprises ROH and M_{A}OH in addition to the products M_{A}OR and water is obtained in step (α1) in the reaction column RR_{A}.

The bottoms product stream S_{AP} comprising ROH and M_{A}OR is then obtained and withdrawn at the lower end of RR_{A}.

A water-containing alcohol stream, presently described as “vapour stream S_{AB} comprising water and ROH”, is withdrawn at the upper end of RR_{A}, preferably at the column top of RR_{A}.

This vapour stream S_{AB} comprising water and ROH is employed in step (β) at least partially as mixture G in step (a) of the process according to the invention. A portion of the alcohol obtained in stream S_{OA} in the distillation in step (a) may be supplied to the reaction column RR_{A} as reactant stream S_{AE1}.

In a preferred embodiment of the process according to the invention a portion of S_{OA} is employed as reactant stream S_{AE1} in step (α1) and if step (α2) is performed alternatively or in addition as reactant stream S_{BE1} in step (α2).

In a more preferred embodiment of the process according to the invention 5% to 95% by weight, preferably 10% to 90% by weight, more preferably 20% to 80% by weight, yet more preferably 30% to 70% by weight, of the vapour stream S_{OA} is employed as reactant stream S_{AE1} or if step (α2) is performed alternatively or in addition as reactant stream S_{BE1} in step (α2).

In this preferred embodiment it is advantageous to compress the portion of the stream S_{OA} employed as reactant stream S_{AE1}/as reactant stream S_{BE1}.

The amount of the alcohol ROH comprised by the reactant stream S_{AE1} is preferably chosen such that said alcohol also serves as a solvent for the alkali metal alkoxide M_{A}OR obtained in the bottoms product stream S_{AP}. The amount of the alcohol ROH in the reactant stream S_{AE1} is preferably chosen to achieve in the bottom of the reaction column the desired concentration of the alkali metal alkoxide solution which is withdrawn as a bottoms product stream S_{AP} comprising ROH and M_{A}OR.

In a preferred embodiment of the process according to the invention, and especially in the cases where S_{AE2} contains water in addition to M_{A}OH, the ratio of the total weight (masses; units: kg) of alcohol employed in step (α1) as reactant stream S_{AE1} ROH to the total weight (masses; unit: kg) of M_{A}OH employed in step (α1) as reactant stream S_{AE2} is 4:1 to 50:1, more preferably 8:1 to 48:1, yet more preferably 10:1 to 45:1, yet still more preferably 20:1 to 40:1.

The reaction column RR_{A} is operated with or without, preferably with, reflux.

“With reflux” is to be understood as meaning that the vapour stream S_{AB}/S_{BB} comprising water and ROH withdrawn at the upper end of the respective column, in step (α1) from the reaction column RR_{A}, in step (α2) from the reaction column RR_{B}, is not completely discharged. Thus in step (β) the respective vapour stream S_{AB}/S_{BB} is then not entirely employed as mixture G but rather at least partially, preferably partially, returned to the respective column as reflux, in step (α1) to the reaction column RR_{A}, in step (α2) to the reaction column RR_{B}. In the cases where such a reflux is established the reflux ratio is preferably 0.01 to 1, more preferably 0.02 to 0.9, yet more preferably 0.03 to 0.34, particularly preferably 0.04 to 0.27 and very particularly preferably 0.05 to 0.24.

A reflux may be established by attaching a condenser at the top of the respective column. In step (α1) this is achieved in particular by attaching a condenser K_{RRA} to the reaction column RR_{A}. In step (α2) this is achieved in particular by attaching a condenser K_{RRB} to the reaction column RR_{B}.

In the respective condenser the respective vapour stream S_{AB}/S_{BB} is at least partially condensed and returned to the respective column, in step (α1) to the reaction column RR_{A}/RR_{B}.

In the embodiment in which a reflux is established on the reaction column RR_{A} the M_{A}OH employed in step (α1) as reactant stream S_{AE2} may also be at least partially mixed with the reflux stream and the resulting mixture thus supplied to step (α1).

Step (α1) is performed especially at a temperature in the range from 45° C. to 150° C., preferably 47° C. to 120° C., more preferably 60° ° C. to 110° C., and at a pressure of 0.5 bar abs. to 40 bar abs., preferably in the range from 0.7 bar abs. to 5 bar abs., more preferably in the range from 0.8 bar abs. to 4 bar abs., more preferably in the range from 0.9 bar abs. to 3.5 bar abs., more preferably at 1.0 bar abs. to 3 bar abs.

The reaction column RR_{A} comprises in a preferred embodiment at least one evaporator which is especially selected from intermediate evaporators V_{ZA} and bottoms evaporators V_{SA}. The reaction column RR_{A} particularly preferably comprises at least one bottoms evaporator V_{SA}.

According to the invention “intermediate evaporators” V_{Z} are to be understood as meaning evaporators arranged above the bottom of the respective column, in particular above the bottom of the reaction column RR_{A}/RR_{B} (then referred to as “V_{ZA}”/“V_{ZB}”) or above the bottom of the rectification column RD_{A} (then referred to as “V_{ZRD}”). In the case of RR_{A}/RR_{B} said evaporators especially evaporate crude product RP_{A}/RP_{B} which is withdrawn from the column as side stream S_{ZAA}/S_{ZBA}.

According to the invention “bottoms evaporators” Vs are to be understood as meaning evaporators which heat the bottom of the respective column, in particular the bottom of the reaction column RR_{A}/RR_{B} or RR_{C} as used in the preferred embodiment and more particularly described hereinbelow (then referred to as “V_{SA}” or “V_{SA}”/“V_{SB}” or “V_{SB}.”/“V_{SC}” or “V_{SC}.”) or the bottom of the rectification column RD_{A} (then referred to as “V_{SRD}” Or “V_{SRD}.”). In the case of RR_{A}/RR_{B} said evaporators especially evaporate at least a portion of the bottoms product stream S_{AP}/S_{BP}. In the case of RR_{C} said evaporators especially evaporate bottoms product stream S_{CP}. In the case of RD_{A} said evaporators especially evaporate bottoms product stream S_{UA} or a portion of S_{UA}, S_{UA1}.

An evaporator is typically arranged outside the respective reaction column or rectification column. Since evaporators transfer energy, in particular heat, from one stream to another they are heat exchangers WT. The mixture to be evaporated is withdrawn via a takeoff from the column and supplied to the at least one evaporator. In the case of the reaction column RR_{A}/RR_{B} intermediate evaporation of the crude product RP_{A}/RP_{B} comprises withdrawal thereof and supply thereof to the at least one intermediate evaporator V_{ZA}/V_{ZB}.

In the case of the rectification column RD_{A} intermediate evaporation comprises withdrawal of at least one side stream S_{ZA} from RD_{A} and supply thereof to the at least one intermediate evaporator V_{ZRD}.

In the case of the rectification column RD_{A} bottoms evaporation comprises withdrawal of at least one stream S_{UA} from RD_{A} and supply of at least a portion, preferably a portion, thereof to the at least one bottoms evaporator V_{SRD}.

The evaporated mixture is recycled back into the respective column optionally with a residual proportion of liquid via at least one feed. When the evaporator is an intermediate evaporator, i.e. in particular an intermediate evaporator V_{ZA}/V_{ZB}/V_{ZRD}, the takeoff by means of which the respective mixture is withdrawn and supplied to the evaporator is a side stream takeoff and the feed by means of which the evaporated mixture is returned to the respective column is a side stream feed. When the evaporator is a bottoms evaporator, i.e. heats the column bottoms, i.e. is in particular a bottoms evaporator V_{SA}/V_{SB}/V_{SRD}, at least a portion of the bottoms takeoff stream, in particular S_{AP}/S_{BP}, is supplied to the bottoms evaporator, evaporated and recycled back into the respective column in the region of the column bottom. However it is alternatively also possible to configure suitable tubes, for example on a suitable tray when using an intermediate evaporator or in the bottom of the respective column, traversed by the heat transfer medium, for example the respective compressed vapour stream S_{OA11}/S_{OA2} (if Vs/V_{Z} are located at the rectification column RD_{A}), or a heating medium W₁ In this case, the evaporation occurs on the tray or in the bottom region of the column. However, it is preferable to arrange the evaporator outside the respective column.

Suitable evaporators employable as intermediate evaporators and bottoms evaporators include for example natural circulation evaporators, forced circulation evaporators, forced circulation evaporators with decompression, kettle evaporators, falling film evaporators or thin film evaporators. Heat exchangers for the evaporator typically employed in the case of natural circulation evaporators and forced circulation evaporators are shell and tube or plate apparatuses. When using a shell and tube exchanger the heat transfer medium, for example the compressed vapour stream S_{OA11}/S_{OA2} in V_{SRD}/V_{ZRD} at the rectification column RD_{A}/the heating medium W₁, may either flow through the tubes with the mixture to be evaporated flowing around the tubes or else the heat transfer medium, for example the compressed vapour stream S_{OA11}/S_{OA2} in V_{SRD}/V_{ZRD} at the rectification column RD_{A}/the heating medium W₁, flows around the tubes with the mixture to be evaporated flowing through the tubes. In the case of a falling-film evaporator, the mixture to be evaporated is typically introduced as a thin film on the inside of a tube and the tube is heated externally. In contrast to a falling-film evaporator, a thin-film evaporator additionally comprises a rotor with wipers which distributes the liquid to be evaporated on the inner wall of the tube to form a thin film.

In addition to the recited evaporator types it is also possible to employ any desired further evaporator type known to those skilled in the art and suitable for use on a rectification column.

When the evaporator operated for example with the compressed vapour stream S_{OA11}/the heating medium W₁ as heating vapour is an intermediate evaporator it is preferable when the intermediate evaporator is arranged in the stripping portion of the rectification column RD_{A} in the region between the feed point of the mixture G and above the column bottom or in the case of the reaction columns RR_{A}/RR_{B} below the feed point of the reactant stream S_{AE2}/S_{BE2}. This makes it possible to introduce a predominant proportion of the heat energy via the intermediate evaporator. It is thus possible for example to introduce more than 80% of the energy via the intermediate evaporator. According to the invention the intermediate evaporator is preferably arranged and/or configured such that it is used to introduce more than 10%, in particular more than 20%, of the total energy required for the distillation.

When using an intermediate evaporator it is especially advantageous when the intermediate evaporator is arranged such that the respective rectification column/reaction column has 1 to 50 theoretical trays below the intermediate evaporator and 1 to 200 theoretical trays above the intermediate evaporator. It is especially preferred when the rectification column/reaction column has 2 to 10 theoretical trays below the intermediate evaporator and 20 to 80 theoretical trays above the intermediate evaporator.

The side stream takeoff by means of which the mixture from the rectification column/reaction column is supplied to the intermediate evaporator V_{Z} and the side stream feed by means of which the evaporated mixture from the intermediate evaporator V_{Z} is returned to the respective rectification column/reaction column may be positioned between the same trays of the column. However, it is also possible for the side stream takeoff and side stream feed to be arranged at different heights.

Such an intermediate evaporator V_{ZA} can convert liquid crude product RP_{A} present in the reaction column RR_{A} comprising M_{A}OR, water, ROH, M_{A}OH into the gaseous state, thus improving the efficiency of the reaction according to step (α1) of the process according to the invention.

Such an intermediate evaporator V_{ZB} can convert liquid crude product RP_{B} present in the reaction column RR_{B} comprising M_{B}OR, water, ROH, M_{B}OH into the gaseous state, thus improving the efficiency of the reaction according to step (α2) of the process according to the invention.

By arranging one or more intermediate evaporators V_{ZA}/V_{ZB} in the upper region of the reaction column RR_{A} the dimensions in the lower region of the reaction column RR_{A} can be reduced. In the embodiment having at least one, preferably two or more, intermediate evaporators V_{ZA}/V_{ZB} it is also possible to introduce substreams of the ROH in liquid form in the upper region of the reaction column RR_{A}.

In a further preferred embodiment energy, preferably heat, is transferred from at least a portion of a stream selected from S_{OA1}, S_{OA2}, in particular selected from S_{OA11}, S_{OA12}, S_{OA2}, preferably selected from S_{OA11}, S_{OA2}, to the crude product RP_{A} and if step (α2) is performed alternatively or in addition to the crude product RP_{B}.

“Transfer of energy, preferably heat, from at least a portion of S_{OA1} to the crude product RP_{A} and if step (α2) is performed alternatively or in addition to the crude product RP_{B}” also comprises the transfer of energy, preferably heat, from at least one stream selected from S_{OA11}, S_{OA12}, or from the stream S_{OA1} before separation thereof into S_{OA11}, S_{OA12}, to the crude product RP_{A} and if step (α2) is performed alternatively or in addition to the crude product RP_{B}. It also comprises the transfer of energy from a portion of S_{OA11}, S_{OA12} to the crude product RP_{A} and if step (α2) is performed alternatively or in addition to the crude product RP_{B}.

To this end especially a portion of the relevant stream selected from S_{OA1}, S_{OA2} or a heat transfer medium W₁, to which energy had previously been transferred from the relevant stream selected from S_{OA1}, S_{OA2}, is at least partially passed through an intermediate evaporator V_{ZA}/V_{ZB} and the energy from the relevant stream selected from S_{OA1}, S_{OA2}/W₁ transferred to the crude product stream withdrawn via the side stream takeoff on RR_{A}/RR_{B}, in particular by utilizing the relevant stream selected from S_{OA1}, S_{OA2}/W₁ for heating the evaporator V_{ZA}/V_{ZB}.

According to the invention bottoms evaporators are arranged at the bottom of the respective rectification column RD_{A}/reaction column RR_{A}/RR_{B}/RR_{C} and are then referred to as “V_{SRD}” or “V_{SRD′}”/“V_{SA}” Or “V_{SA′}”/“V_{SB}” Or “V_{SB′}”/“V_{SC}” or “V_{SC′}”. A bottoms product stream (in particular S_{AP}/S_{BP}) present in the respective column (in particular reaction column RR_{A}/RR_{B}) may be passed into such a bottoms evaporator and for example ROH at least partially removed therefrom. In the case of S_{AP}/S_{BP} this may afford a bottoms product stream S_{AP*} having an elevated mass fraction of M_{A}OR compared to S_{AP} or a bottoms product stream S_{BP*} having an elevated mass fraction of M_{B}OR compared to S_{BP}.

In step (α1) of the process according to the invention a bottoms product stream S_{AP} comprising ROH and M_{A}OR is withdrawn at the lower end of the reaction column RR_{A}.

It is preferable when the reaction column RR_{A} comprises at least one bottoms evaporator V_{SA} through which the bottoms product stream S_{AP} is then partially passed to partially remove ROH, thus affording a bottoms product stream S_{AP*} having an elevated mass fraction of M_{A}OR compared to S_{AP}.

In another preferred embodiment transfer of energy, preferably heat, from at least a portion of a stream selected from S_{OA1}, S_{OA2}, in particular selected from S_{OA11}, S_{OA2}, to the crude product RP_{A} and if step (α2) is performed alternatively or in addition to the crude product RP_{B} is therefore undertaken as follows:

Especially a portion of the relevant stream selected from S_{OA1}, S_{OA2} or a heat transfer medium W₁, to which energy had previously been transferred from the relevant stream selected from S_{OA1}, S_{OA2}, is then at least partially passed through a bottoms evaporator V_{SA}/V_{SB} and the energy from the relevant stream selected from S_{OA1}, S_{OA2}/W₁ transferred to the bottoms product stream S_{AP}/S_{BP}, in particular by utilizing the relevant stream selected from S_{OA1}, S_{OA2}/W₁ for heating the evaporator V_{SA}/V_{SB}.

The mass fraction of M_{A}OR in the bottoms product stream S_{AP*} is in particular elevated compared to the mass fraction of M_{A}OR in the bottoms product stream S_{AP} by at least 0.5%, preferably by ≥ 1%, more preferably by ≥2%, yet more preferably by ≥5%.

It is preferable when S_{AP} or, if at least one bottoms evaporator V_{SA} is used through which the bottoms product stream S_{AP} is at least partially passed to at least partially remove ROH, S_{AP*} has a mass fraction of M_{A}OR in ROH in the range from 1% to 50% by weight, preferably 5% to 35% by weight, more preferably 15% to 35% by weight, most preferably 20% to 35% by weight, in each case based on the total mass of S_{AP}.

The mass fraction of residual water in S_{AP}/S_{AP*} is preferably <1% by weight, preferably <0.8% by weight, more preferably <0.5% by weight, based on the total mass of S_{AP}.

The mass fraction of reactant M_{A}OH in S_{AP}/S_{AP*} is preferably <1% by weight, preferably <0.8% by weight, more preferably <0.5% by weight, based on the total mass of S_{AP}.

### 4.2.2 Step (α2) (Optional)

Step (α2) is an optional embodiment of the process according to the invention. This is to be understood as meaning that in the context of the preferred embodiment of the process according to the invention step (α2) is or is not performed. In optional step (α2), simultaneously with and spatially separate from step (α1), a reactant stream S_{BE1} comprising ROH is reacted with a reactant stream S_{BE2} comprising M_{B}OH in countercurrent in a reactive rectification column RR_{B} to afford a crude product RP_{B} comprising M_{B}OR, water, ROH, M_{B}OH.

In step (α2) of the process according to the invention a bottoms product stream S_{BP} comprising ROH and M_{B}OR is withdrawn at the lower end of RR_{B}. A vapour stream S_{BB} comprising water and ROH is withdrawn at the upper end of RR_{B}.

M_{B} is selected from sodium, potassium, preferably potassium.

The reactant stream S_{BE1} comprises ROH. In a preferred embodiment the mass fraction of ROH in S_{BE1} is ≥95% by weight, yet more preferably ≥99% by weight, wherein S_{BE1} otherwise comprises especially water.

The alcohol ROH employed in step (α2) of the process according to the invention as reactant stream S_{BE1} may also be commercially available alcohol having an alcohol mass fraction of more than 99.8% by weight and a mass fraction of water of up to 0.2% by weight.

The reactant stream S_{BE1} is preferably introduced in vapour form.

The reactant stream S_{BE2} comprises M_{B}OH. In a preferred embodiment S_{BE2} comprises not only M_{B}OH but also at least one further compound selected from water, ROH. It is yet more preferable when S_{BE2} comprises water in addition to M_{B}OH, thus rendering S_{BE2} an aqueous solution of M_{B}OH.

When the reactant stream S_{BE2} comprises M_{B}OH and water the mass fraction of M_{B}OH based on the total weight of the aqueous solution forming S_{BE2} is especially in the range from 10% to 75% by weight, preferably from 15% to 54% by weight, more preferably from 30% to 53% by weight and particularly preferably from 40% to 52% by weight.

When the reactant stream S_{BE2} comprises M_{B}OH and ROH the mass fraction of M_{B}OH in ROH based on the total weight of the solution forming S_{BE2} is especially in the range from 10% to 75% by weight, preferably from 15% to 54% by weight, more preferably from 30% to 53% by weight and particularly preferably from 40% to 52% by weight.

In the particular case in which the reactant stream S_{BE2} comprises both water and ROH in addition to M_{B}OH it is particularly preferable when the mass fraction of M_{B}OH in ROH and water based on the total weight of the solution forming S_{BE2} is especially in the range from 10% to 75% by weight, preferably from 15% to 54% by weight, more preferably from 30% to 53% by weight and particularly preferably from 40% to 52% by weight.

Step (α2) of the process according to the invention is performed in a reactive rectification column (or “reaction column”) RR_{B}. Preferred embodiments of the reaction column RR_{B} are described in section 4.2.1.

“Reaction of a reactant stream S_{BE1} comprising ROH with a reactant stream S_{BE2} comprising M_{B}OH in countercurrent” is according to the invention achieved, in particular, as a result of the feed point for at least a portion of the reactant stream S_{BE1} comprising ROH in step (α2) being located on the reaction column RR_{B} below the feed point of the reactant stream S_{BE2} comprising M_{B}OH.

The reaction column RR_{B} preferably comprises at least 2, in particular 15 to 40, theoretical trays between the feed point of the reactant stream S_{BE1} and the feed point of the reactant stream S_{BE2}.

The reaction column RR_{A} may be operated as a pure stripping column. In this case the reactant stream S_{BE1} comprising ROH is introduced in vapour form in the lower region of the reaction column RR_{B}. Step (α2) of the process according to the invention also encompasses the case where a portion of the reactant stream S_{BE1} comprising ROH is added in vapour form below the feed point of the reactant stream S_{BE2} comprising aqueous sodium hydroxide solution M_{B}OH but nevertheless at the upper end or in the region of the upper end of the reaction column RR_{B}. This makes it possible to reduce the dimensions of the lower region of the reaction column RR_{B}. When a portion of the reactant stream S_{BE1} comprising ROH, in particular methanol, is added especially in vapour form at the upper end or in the region of the upper end of the reaction column RR_{B} only a fraction of 10% to 70% by weight, preferably of 30% to 50% by weight, (in each case based on the total amount of the alcohol ROH employed in step (α2)) is introduced at the lower end of the reaction column RR_{B} and the remaining fraction is added in vapour form in a single stream or divided into a plurality of substreams preferably 1 to 10 theoretical trays, particularly preferably 1 to 3 theoretical trays, below the feed point of the reactant stream S_{BE2} comprising M_{B}OH.

In the reaction column RR_{B} the reactant stream S_{BE1} comprising ROH is then reacted with the reactant stream S_{BE2} comprising M_{B}OH according to the reaction <1> described hereinabove to afford M_{B}OR and H₂O, where these products are present in admixture with the reactants ROH and M_{B}OH since an equilibrium reaction is concerned. Accordingly a crude product RP_{B} which contains not only the products M_{B}OR and water but also ROH and M_{B} is obtained in the reaction column RR_{B} in step (α2) of the process according to the invention.

The bottom product stream S_{BP} comprising ROH and M_{B}OR is obtained and withdrawn at the lower end of RR_{B}.

A water-containing alcohol stream, presently described as “vapour stream S_{BB} comprising water and ROH”, is withdrawn at the upper end of RR_{B}, preferably at the column top of RR_{B}.

In step (β) of the process according to the invention at least a portion of this vapour stream S_{BB} comprising water and ROH is employed especially as mixture G in step (a) of the process according to the invention. Said mixture is supplied to the rectification column RD_{A} as mixture G in admixture with S_{AB} or not, i.e. separate from S_{AB}. It is preferable when the vapour streams S_{BB} and S_{AB} are mixed and then the mixture is employed as mixture G in step (a) of the process according to the invention.

The amount of alcohol ROH present in the reactant stream S_{BE1} is preferably selected so that it simultaneously serves as solvent for the alkali metal alkoxide M_{B}OR present in the bottom product stream S_{BP}. The amount of the alcohol ROH in the reactant stream S_{BE1} is preferably chosen to achieve in the bottom of the reaction column the desired concentration of the alkali metal alkoxide solution which is withdrawn as a bottoms product stream S_{BP} comprising ROH and M_{B}OR.

In a preferred embodiment of the process according to the invention, and especially in the cases where S_{BE2} contains water in addition to M_{B}OH, the ratio of the total weight (masses; units: kg) of alcohol employed in step (α2) as reactant stream S_{BE1} ROH to the total weight (masses; unit: kg) of M_{B}OH employed in step (α2) as reactant stream S_{BE2} is 4:1 to 50:1, more preferably 8:1 to 48:1, yet more preferably 10:1 to 45:1, yet still more preferably 20:1 to 40:1.

The reaction column RR_{B} is operated with or without, preferably with, reflux.

“With reflux” is to be understood as meaning that the vapour stream S_{BB} withdrawn at the upper end of the respective column, the reaction column RR_{B} in step (α2), comprising water and ROH is not completely discharged, i.e. in step (β) of the process according to the invention not completely employed as mixture G in step (a), i.e. supplied to the rectification column RD_{A}, but rather at least partially, preferably partially, returned to the respective column, the reaction column RR_{B} in step (α2), as reflux. In the cases where such a reflux is established the reflux ratio is preferably 0.01 to 0.99, more preferably 0.02 to 0.9, yet more preferably 0.03 to 0.34, particularly preferably 0.04 to 0.27 and very particularly preferably 0.05 to 0.24.

In the embodiment in which a reflux is established for the reaction column RR_{B} the M_{B}OH employed in step (α2) as reactant stream S_{BE2} may also be at least partially mixed with the reflux stream and the resulting mixture thus supplied to step (α2).

Optional step (α2) is performed especially at a temperature in the range from 45° C. to 150° C., preferably 47° C. to 120° C., more preferably 60° ° C. to 110° C., and at a pressure of 0.5 bar abs. to 40 bar abs. preferably in the range from 0.7 bar abs. to 5 bar abs., more preferably in the range from 0.8 bar abs. to 4 bar abs., more preferably in the range from 0.9 bar abs. to 3.5 bar abs., yet more preferably at 1.0 bar abs. to 3 bar abs.

The reaction column RR_{B} comprises in a preferred embodiment at least one evaporator which is in particular selected from intermediate evaporators V_{ZB} and bottoms evaporators V_{SB}. The reaction column RR_{B} particularly preferably comprises at least one bottoms evaporator V_{SB}.

Such an intermediate evaporator V_{ZB} can convert liquid crude product RP_{B} present in the reaction column RR_{B} comprising M_{B}OR, water, ROH, M_{B}OH into the gaseous state, thus improving the efficiency of the reaction according to step (α2) of the process according to the invention.

By arranging one or more intermediate evaporators V_{ZB} in the upper region of the reaction column RR_{B} the dimensions in the lower region of the reaction column RR_{B} can be reduced. In the embodiment having at least one, preferably two or more, intermediate evaporators V_{ZB} it is also possible to introduce substreams of the ROH in liquid form in the upper region of the reaction column RR_{B}.

In step (α2) of the process according to the invention a bottoms product stream S_{BP} comprising ROH and M_{B}OR is withdrawn at the lower end of the reaction column RR_{B}.

It is preferable when the reaction column RR_{B} comprises at least one bottoms evaporator V_{SB} through which the bottoms product stream S_{BP} is then at least partially passed to at least partially remove ROH, thus affording a bottoms product stream S_{BP*} having an elevated mass fraction of M_{B}OR compared to S_{BP}.

The mass fraction of M_{B}OR in the bottoms product stream S_{BP*} is in particular elevated compared to the mass fraction of M_{B}OR in the bottoms product stream S_{BP} by at least 0.5%, preferably by ≥1%, more preferably by ≥2%, yet more preferably by ≥5%.

It is preferable when S_{BP} or, if at least one bottoms evaporator V_{SB} is employed through which the bottoms product stream S_{BP} is at least partially passed to at least partially remove ROH, S_{BP*} has a mass fraction of M_{B}OR in ROH in the range from 1% to 50% by weight, preferably 5% to 35% by weight, more preferably 15% to 35% by weight, most preferably 20% to 35% by weight, in each case based on the total mass of S_{BP}.

The mass fraction of residual water in S_{BP}/S_{BP*} is preferably <1% by weight, preferably <0.8% by weight, more preferably <0.5% by weight, based on the total mass of S_{BP}.

The mass fraction of reactant M_{B}OH in S_{BP}/S_{BP*} is preferably <1% by weight, preferably <0.8% by weight, more preferably <0.5% by weight, based on the total mass of S_{BP}.

In the embodiments of the present process in which step (α2) is also performed it is preferable when the bottoms product stream S_{AP} is at least partially passed through a bottoms evaporator V_{SA} and ROH is at least partially removed from S_{AP} to afford a bottoms product stream S_{AP*} having an elevated mass fraction of M_{A}OR compared to S_{AP} and/or, preferably and, the bottoms product stream S_{BP} is at least partially passed through a bottoms evaporator V_{SB} and ROH is at least partially removed from S_{BP} to afford a bottoms product stream S_{BP*} having an elevated mass fraction of M_{B}OR compared to S_{BP}.

In the embodiments of the present invention in which it is performed step (α2) of the process according to the invention is performed simultaneously with and spatially separate from step (α1). Spatial separation is ensured by performing steps (α1) and (α2) in the two reaction columns RR_{A} and RR_{B}.

In an advantageous embodiment of the invention the reaction columns RR_{A} and RR_{B} are accommodated in one column shell, where the column is at least partially subdivided by at least one dividing wall. Such a column having at least one dividing wall will be referred to as “TRD”. Such dividing wall columns are familiar to those skilled in the art and are described for example in U.S. Pat. No. 2,295,256, EP 0 122 367 A2, EP 0 126 288 A2, WO 2010/097318 A1 and by I. Dejanović, Lj. Matijašević, Ž. Olujić, Chemical Engineering and Processing 2010, 49, 559-580. CN 105218315 A likewise describes dividing wall columns which are used in the rectification of methanol.

In the dividing wall columns suitable for the process according to the invention the dividing walls preferably extend to the column floor and, in particular, preferably span at least a quarter, more preferably at least a third, yet more preferably at least half, yet more preferably at least two thirds, yet still more preferably at least three quarters, of the column by height. They divide the columns into at least two reaction spaces in which spatially separate reactions may be carried out. The reaction spaces provided by the at least one dividing wall may be of identical or different sizes.

In this embodiment the bottoms product streams S_{AP} and S_{BP} may be separately withdrawn in the respective regions separated by the dividing wall and preferably passed through the bottoms evaporator V_{SA}/V_{SB} attached for each reaction space formed by the at least one reaction wall in which ROH is at least partially removed from S_{AP}/S_{BP} to afford S_{AP*}/S_{BP*}.

In a preferred embodiment of the process according to the invention accordingly at least two, more preferably precisely two, of the columns selected from rectification column RD_{A}, reaction column RR_{A} and if step (α2) is performed the reaction column RR_{B} are accommodated in one column shell, wherein the columns are at least partially separated from one another by a dividing wall extending to the bottom of the column.

In the integrated system comprising reaction column RR_{A} (or in the embodiments in which step (α2) is performed reaction column RR_{A} and reaction column RR_{B}) and rectification column RD_{A} in the process according to the invention the rectification column RD_{A} is preferably operated at a pressure selected such that the pressure gradient between the columns is low.

The alcohol ROH is consumed in the process according to the invention and especially in a continuous process mode therefore requires replacement with fresh alcohol ROH.

Supply of the fresh alcohol ROH is thus especially carried out directly as reactant stream S_{AE1} comprising ROH into the reaction column RR_{A} or in the embodiments in which step (α2) is performed into the reaction columns RR_{A} and RR_{B}.

In the process according to the invention it is further preferable to employ the ROH-comprising vapour stream S_{OA} partially as reactant stream S_{AE1} in step (α1) and optionally as reactant stream S_{BE1} in step (α2). The compressed vapour stream S_{OA1} may alternatively or in addition be employed partially as reactant stream S_{AE1} in step (α1) and optionally as reactant stream S_{BE1} in step (α2). In this preferred embodiment it is yet more preferable when the fresh alcohol ROH is added to the rectification column RD_{A}.

When the fresh alcohol ROH is added to the rectification column RD_{A} it is preferably supplied either in the rectifying section of the rectification column RD_{A} or directly at the top of the rectification column RD_{A}. The optimal feed point depends on the water content of the employed fresh alcohol and also on the desired residual water content in the vapour stream S_{OA}. The higher the proportion of water in the employed alcohol and the higher the purity requirements of the vapour stream S_{OA} the more advantageous is a feed a number of theoretical plates below the top of the rectification column RD_{A}. Up to 20 theoretical plates below the top of the rectification column RD_{A} and in particular 1 to 5 theoretical plates are preferred.

When the fresh alcohol ROH is added to the rectification column RD_{A} it is added at the top of the rectification column RD_{A} at temperatures up to boiling point, preferably at room temperature. A dedicated feed may be provided for the fresh alcohol or else when a portion of the alcohol withdrawn at the top of the rectification column RD_{A} is recycled may be mixed therewith after condensation and supplied to the rectification column RD_{A} together. In this case it is particularly preferable when the fresh alcohol is added to a condensate container in which the alcohol condensed from the vapour stream S_{OA} is collected.

As described hereinabove, in an advantageous embodiment of the invention at least two of the columns selected from rectification column RD_{A}, reaction column RR_{A} and if step (α2) is performed the reaction column RR_{B} are accommodated in one column shell, wherein the columns are in each case at least partially separated from one another by a dividing wall extending to the bottom of the column. In the abovedescribed preferred embodiment in which step (α2) is performed these are thus separated from one another by two dividing walls, wherein the two dividing walls extend to the bottom of the column.

In this preferred embodiment the reaction to afford the crude product RP_{A} according to step (α1) or the crude products RP_{A} and RP_{B} according to steps (α1) and (α2) are in particular performed in one part of the TRD, wherein the reactant stream S_{AE2} and optionally the reactant stream S_{BE2} are added below but at approximately the height of the upper end of the dividing wall and the reactant stream S_{AE1} and optionally the reactant stream S_{BE1} are added in vapour form at the lower end. The alcohol/water mixture formed above the feed point of the reactant stream then becomes distributed above the dividing wall over the entire column region which serves as rectifying section of the rectification column RD_{A}. The second/third lower part of the column which has been separated off by the dividing wall is the stripping section of the rectification column RD_{A}. The energy required for the distillation is then supplied via an evaporator at the lower end of the second portion of the column separated by the dividing wall, wherein this evaporator may be conventionally heated or heated with a portion of the compressed vapour stream S_{OA2}. When the evaporator is conventionally heated an intermediate evaporator heated with a portion of the compressed vapour stream S_{OA11} may additionally be provided.

In the embodiments in which a portion of S_{OA} is employed as reactant stream S_{AE1} and/or reactant stream S_{BE1} S_{OA} is especially compressed with a first compressor VD_{AB2} (“precompressed”), wherein the difference in the pressures inside the reaction columns RR_{A} and RR_{B} compared to the pressure in RD_{A} may be taken into account.

Instead of the compressor VD_{AB2} which is arranged downstream of the rectification column RD_{A} and in which S_{OA} is precompressed this preferred embodiment may also employ, alternatively or in addition, a compressor VD_{AB1} which is arranged upstream of the rectification column RD_{A} and through which the mixture G is passed before it is passed into RD_{A}.

Provided it is not recycled to RD_{A} as reflux the remaining portion of the at least one vapour stream S_{OA}, i.e. especially the portion not employed as reactant stream S_{AE1} and/or reactant stream S_{BE1}, is in this preferred embodiment then further compressed to afford S_{OA1}. According to the invention S_{OA} becomes S_{OA1} only in the compressor stage after which S_{OA1} is divided into S_{OA11} and S_{OA12} as are employed in step (d) according to the invention. This compression to compress S_{OA} into S_{OA1} is in the figures and examples performed with the compressor V_{D1} (referred to in the figures as <401>).

### 4.2.3 Step (β)

In step (B) of the process according to the invention at least a portion of the vapour stream S_{AB}, and if step (α2) is performed at least a portion of the vapour stream S_{BB}, in admixture with S_{AB} Or separate from S_{AB}, is employed as mixture G in step (a) of the process according to the invention. If step (α2) is performed it is preferable when the at least a portion of the vapour stream S_{AB} and the at least a portion of the vapour stream S_{BB} are mixed and then employed as mixture G in step (a) of the process according to the invention.

“Employed as mixture G in step (a) of the process according to the invention” is in particular to be understood as meaning that the two streams S_{AB} and S_{BB} are passed into the rectification column RD_{A} and are preferably previously mixed.

However, they may alternatively also be passed into the rectification column RD_{A} at two different feed points.

### 4.3 Preferred Aspect: Process for Transalcoholization of an Alkali Metal Alkoxide

In an advantageous embodiment of the present invention the energy comprised in at least one of the streams S_{OA1}, S_{OA2}, S_{OA11}, S_{OA12} is used for operation of other industrial processes. This is advantageous especially in sites hosting integrated systems (chemistry parks, technology parks) where there is always a need for heat for heating. Especially in the case of integrated systems comprising two or more plants for alkali metal alkoxide production this energy may be advantageously utilized. Such integrated systems typically also comprise processes for transalcoholization as described in DE 27 26 491 A1. U.S. Pat. No. 3,418,383 A describes processes for transalcoholization from methoxides to propoxides.

A preferred aspect of the present invention provides that in the process according to the present invention in a reactive rectification column RR_{C} a reactant stream S_{CE1} comprising M_{c}OR′ and optionally R′OH is reacted in countercurrent with a reactant stream S_{CE2} comprising R″OH to afford a crude product RP_{C} comprising M_{c}OR″ and R′OH, wherein a bottoms product stream S_{CP} comprising M_{c}OR″ is withdrawn at the lower end of RR_{C} and a vapour stream S_{CB} comprising R′OH is withdrawn at the upper end of RR_{C},
and wherein R′ and R″ are two distinct C₁ to C₆ hydrocarbon radicals and M_{C} is a metal selected from sodium, potassium, preferably sodium,
and wherein energy is transferred from at least a portion of a stream selected from S_{OA1}, S_{OA2} to the crude product RR_{C}.

The process according to the invention according to the preferred aspect of the invention is a process for transalcoholization of a particular alkali metal alkoxide M_{c}OR′ to afford another alkali metal alkoxide M_{c}OR″ as described for example in DE 27 26 491 A1.

R′ and R″ are two distinct C₁ to C₆ hydrocarbon radicals, preferably two distinct C₁ to C₄ hydrocarbon radicals.

More preferably R′ is methyl and R″ is a C₂ to C₄ hydrocarbon radical, yet more preferably R′=methyl and R″=ethyl.

The process according to the preferred aspect of the invention (hereinbelow also referred to as “transalcoholization”) is performed in a reactive rectification column RR_{C}. Suitable reactive rectification columns include columns described for RR_{A} at point 4.2.1 in the context of step (α1).

The reaction column RR_{C} is operated with or without, preferably with, reflux. If a reflux is established in particular the vapour S_{CB} is partially or completely passed through a condenser K_{RRC} and the condensed vapour may then be returned to the reaction column RR_{C} or employed as reactant stream S_{AE1} Or S_{BE1}. It may also be employed as the fresh alcohol stream in RD_{A}.

In the transalcoholization a bottoms product stream S_{CP} comprising M_{c}OR″ is withdrawn at the lower end of RR_{C}. A vapour stream S_{CB} comprising R′OH is withdrawn at the upper end of RR_{C}.

Preferably, in the embodiments of the transalcoholization which also comprise performing the process for producing an alkali metal alkoxide according to the invention the employed reactant stream S_{CE1} comprising M_{c}OR′ and optionally R′OH preferably comprises at least a portion of S_{AP}, in particular since R=methyl and thus also R′=methyl. It is then particularly preferable when R″=ethyl. A transalcoholization of alkali metal methoxide to the corresponding alkali metal ethoxide is accordingly carried out.

In a preferred alternative in the embodiments of the transalcoholization which also comprise performing the process for producing an alkali metal alkoxide including step (α2) according to the invention the employed reactant stream S_{CE1} comprising M_{c}OR′ and optionally R′OH preferably comprises at least a portion of S_{BP}, in particular since R=methyl and thus also R′=methyl. It is then particularly preferable when R″=ethyl. A transalcoholization of alkali metal methoxide to the corresponding alkali metal ethoxide is accordingly carried out.

When S_{BP} and S_{AP} comprise the same alkali metal alkoxide and the same alcohol ROH these two streams may also be employed separately or in admixture as S_{CE1}, i.e. in particular first mixed and then supplied to the column RR_{C} as reactant stream S_{CE1} or supplied to the column RR_{C} separately as two reactant streams S_{CE1}.

The reactant stream S_{CE2} comprises R″OH. In a preferred embodiment the mass fraction of R″OH in S_{CE2} is ≥85% by weight, more preferably ≥90% by weight, wherein S_{CE2} otherwise comprises in particular M_{c}OR″ or another denaturant. The alcohol R″OH employed as reactant stream S_{CE2} may also be commercially available alcohol having an alcohol mass fraction of more than 99.8% by weight and a mass fraction of water of up to 0.2% by weight.

“Reaction of a reactant stream S_{CE1} comprising M_{c}OR′ and optionally R′OH with a reactant stream S_{CE2} comprising R″OH in countercurrent” is according to the invention ensured, in particular, as a result of the feed point for at least a portion of the reactant stream S_{CE1} comprising M_{c}OR′ being located on the reaction column RR_{C} above the feed point of the reactant stream S_{CE2} comprising R″OH.

The reaction column RR_{C} is operated with or without, preferably with, reflux.

In a preferred embodiment the reaction column RR_{C} comprises at least one evaporator which is in particular selected from intermediate evaporators V_{ZC} and bottoms evaporators V_{SC}. The reaction column RR_{C} particularly preferably comprises at least one bottoms evaporator V_{SC}.

In the case of the reaction column RR_{C} intermediate evaporation comprises withdrawal of at least one side stream S_{ZC} from RR_{C} and supply thereof to the at least one intermediate evaporator V_{ZC}.

In the case of the reaction column RR_{C} bottoms evaporation comprises withdrawal of at least one stream, for example S_{CP} from RR_{C} and supply of at least a portion, in the case of S_{CP} preferably a portion, to the at least one bottoms evaporator V_{SC}.

Suitable evaporators employable as intermediate evaporators and bottoms evaporators are described in section 4.2.1.

In the transalcoholization, energy, preferably heat, is transferred from at least a portion of a stream selected from S_{OA1}, S_{OA2} to the crude product RP_{C}. This is preferably effected by transfer of energy from at least a portion of a stream selected from S_{OA1}, S_{OA2} to S_{CE1} or S_{CE2} before passage thereof into RR_{C} followed by transfer of energy from S_{CE1}/S_{CE2} to the crude product RP_{C} present in RR_{C}, with which they mix together.

Accordingly energy, preferably heat, from at least a portion of a stream selected from S_{OA1}, S_{OA2}, in particular from at least one stream selected from S_{OA11}, S_{OA12}, S_{OA2}, preferably from at least one stream selected from S_{OA11}, a portion of S_{OA2}, is transferred to the crude product RP_{C}.

“Transfer of energy, preferably heat, from at least a portion of S_{OA1} to the crude product RP_{C}″ also comprises the transfer of energy, preferably heat, from at least one stream selected from S_{OA11}, S_{OA12}, stream S_{OA1} before separation thereof into S_{OA11}, S_{OA12} to the crude product RP_{C}.

In addition crude product RP_{C} may also be passed through an intermediate evaporator V_{ZC} or a bottoms evaporator V_{SC} and in V_{ZC}/V_{SC} energy, preferably heat, may be transferred from at least a portion of a stream selected from S_{OA1}, S_{OA2} to the crude product RP_{C}.

In addition the bottoms product stream S_{CP} may also be partially passed through an intermediate evaporator V_{SC} and then partially recycled to RR_{C}, wherein in V_{SC} energy, preferably heat, is transferred from at least a portion of a stream selected from S_{OA1}, S_{OA2} to the recycled portion of S_{CP} and then in the column RR_{C} transferred from S_{CP} to the crude product RP_{C} present in the column.

The transfer of energy from at least a portion of a stream selected from S_{OA1}, S_{OA2} to the recited streams is effected directly or indirectly, i.e. without or with heat transfer medium W₁, as described accordingly in section 4.1.4.

The preferred embodiment of the process according to the invention makes it possible to efficiently employ the energy from S_{OA1}, S_{OA2}, in particular from S_{OA2}, S_{OA11}, S_{OA12}. This reduces the total energy demand.

### 5. EXAMPLES

### 5.1 Example 1 (Noninventive), Corresponds to FIG. 1

A stream of aqueous NaOH (50% by weight) S_{AE2} <102> of 100 kg/h is supplied at 30° C. to the top of a reaction column RR_{A} <100>. A vaporous methanol stream S_{AE1} <103> of 1034.9 kg/h is supplied in countercurrent at the bottom of the reaction column RR_{A} <100>. The reaction column RR_{A} <100> is operated at a head pressure of 2.15 bar abs. At the bottom of the column RR_{A} <100> a virtually water-free product stream S_{AP*} <104> of 219.7 kg/h is withdrawn (30% by weight sodium methoxide in methanol). The evaporator V_{SA} <105> of the reaction column RR_{A} <100> introduces about 24 KW of heating power using low pressure steam. A vaporous methanol-water stream S_{AB} <107> is withdrawn at the top of the reaction column RR_{A} <100> and 80 kg/h thereof are condensed in the condenser K_{RRA} <108> and returned to the reaction column RR_{A} <100> as reflux while the remaining stream of 915.2 kg/h is supplied to a rectification column RD_{A} <300>. The rectification column RD_{A} <300> is operated at a head pressure of 2.0 bar abs. At the bottom of the rectification column RD_{A} <300> a liquid water stream S_{UA} <304> of 72.2 kg/h is discharged (500 ppmw of methanol). At the top of the rectification column RD_{A} <300> a vaporous methanol stream S_{OA} <302> (2 bar, 83° ° C.; 200 ppmw of water) of 1903.6 kg/h is withdrawn and 63.9 kg/h thereof are condensed in a condenser K_{RD} <407> while the remaining stream is supplied to a first compressor VD_{AB2} <303> and therein compressed to 2.6 bar abs. The stream is subsequently divided and a stream of 1034.9 kg/h is recycled to the reaction column RR_{A} <100>. The remainder of 804.8 kg/h is supplied to a multi-stage compression with intermediate cooling. In the compressor V_{D1} <401> the stream is compressed to p_{OA1}=4.8 bar abs. and T_{OA1}=156° C. to obtain stream <403>. In the subsequent intermediate cooling in the intermediate cooler WT_{X} <402> the stream is cooled to 145° C. and about 4.4 KW of heat are removed using cooling water. In the compressor VDₓ <405> the stream is finally compressed again to 9.0 bar and 200° C. to obtain stream <404>. The subsequent condenser which is simultaneously the bottoms evaporator V_{SRD} <406> of the rectification column RD_{A} <300> provides the approximately 238 KW of heating power for the rectification column RD_{A} <300>. The thus condensing methanol stream <404> is mixed together with 191.9 kg/h of fresh methanol (1000 ppmw of water) <408> and the 63.9 kg/h of previously condensed vapours and recycled to the top of the rectification column RD_{A} <300>.

The compressor power sums to about 55 kW.

Together with the 24 KW for the heating steam this results in a compressor and heating steam power demand of about 79 kW.

### 5.2 Example 2 (Noninventive), Corresponds to FIG. 2

The arrangement in noninventive example 2 corresponds to that of example 1 with the following differences:

The rectification column RD_{A} <300> comprises an intermediate evaporator V_{ZRD} <409>. A liquid stream S_{ZA} <305> is withdrawn from the rectification column RD_{A} <300> at 94° C. and about 230 KW of heat are transferred thereto in the intermediate evaporator V_{ZRD} <409>, wherein the stream is partially evaporated and subsequently returned to the rectification column RD_{A} <300>. A vaporous methanol stream S_{OA} <302> (200 ppmw of water) of 1887.1 kg/h is withdrawn at the top of the rectification column RD_{A} <300> and 89.4 kg/h thereof are condensed in a condenser K_{RD} <407>. The remaining stream is compressed to 2.6 bar abs. in a first compressor VD_{AB2} <303> as in example 1. A substream of 1034.9 kg/h is then recycled to the reaction column RR_{A} <100>. The remainder of 762.8 kg/h is compressed to 5.6 bar abs. and 168° C. to obtain stream <403>. The subsequent condenser which is simultaneously the intermediate evaporator V_{SRD} <409> of the rectification column RD_{A} <300> provides the approximately 230 KW of heating power for the rectification column RD_{A} <300>. The thus condensed methanol stream <403> is mixed with 191.9 kg/h of fresh methanol <408> and the 89.4 kg/h of previously condensed vapours and returned to the top of the rectification column RD_{A} <300>. The bottoms evaporator V_{SRD} <406> of the reaction column RD_{A} <300> introduces about 20 KW of heating power using low pressure steam. Compared to example 1 the vapour stream need be compressed only to 5.6 bar abs. instead of 9 bar abs. to be able to transmit its heat to the evaporator V_{ZRD} <409> since the boiling temperature in the intermediate evaporator V_{ZRD} <409> is lower than in the bottoms evaporator V_{SRD} <406>. The compressor power thus sums to only about 38 KW (instead of 55 KW) while the heating steam demand increases relative to example 1 to about 44 KW since the bottoms evaporator V_{SRD} <406> requires a heating power of 20 KW and this introduced with the heat of low pressure steam. The compressor and heating steam power demands thus sum to about 82 kW.

### 5.3 Example 3 Inventive), Corresponds to FIG. 3

The arrangement in inventive example 3 corresponds to that of example 1 and 2 with the following differences:
A vaporous methanol stream S_{OA} <302> (200 ppmw of water) of 1898.9 kg/h is withdrawn at the top of the rectification column RD_{A} <300> and 33.9 kg/h thereof are condensed in a condenser K_{RD} <407>. The remaining stream is compressed to 2.6 bar abs. in a first compressor VD_{AB2} <303> as in example 2 and subsequently a substream of 1034.9 kg/h is recycled to the reaction column RR_{A} <100>. The remainder of 830.1 kg/h is initially compressed to 5.6 bar abs. and 169° C. to obtain stream S_{OA1} <403>. A portion S_{OA11} <4031> (761.7 kg/h) of this stream is passed into a downstream condenser which is simultaneously the intermediate evaporator V_{ZRD} <409> of the rectification column RD_{A} <300> and provides about 230 KW of heating power for the rectification column RD_{A} <300>. The other portion S_{OA12} <4032> (68.4 kg/h) is subsequently cooled to about 154° C. in an intermediate cooling in intermediate cooler WT_{X} <402> wherein about 0.5 KW of heat are removed via cooling water. The stream S_{OA12} <4032> is subsequently compressed in a further compressor VDₓ <405> to obtain stream S_{OA2} <404> having p_{OA2}=9.0 bar and T_{OA}2=196° C. The subsequent condenser which is simultaneously the bottoms evaporator V_{SRD} <406> of the rectification column RD_{A} <300> provides about 20 KW of heating power for the rectification column RD_{A} <300>. The methanol streams S_{OA11} <4031> and S_{OA2} <404> condensed in the intermediate and bottoms evaporators V_{ZRD} <409> and V_{SRD} <406> are mixed with 191.9 kg/h of fresh methanol <408> and the 33.9 kg/h of previously condensed vapours and returned to the top of the rectification column RD_{A} <300>.

The compressor power sums to about 42 KW (instead of 55 KW in example 1). Since no low pressure steam is required for the bottoms evaporator V_{SRD} <406> only about 24 KW need be provided using heating steam as in example 1. The sum of the compressor and heating steam power demands thus falls to about 66 kW.

Compared to example 1 only a lower vapour stream need be compressed to 9 bar abs., a large part of the vapour stream need only be compressed to 5.6 bar abs. as in example 2, thus reducing the total compressor power. However, compared to example 2 steady state operation does not require any low pressure steam in the bottoms evaporator V_{SRD} <406>, thus reducing the heating steam demand compared to example 2.

The total energy to be supplied is minimized by the process according to example 3.

The proportion of heating power provided by low pressure steam and compressor power in each case is shown in FIG. 9.

Result: The inventive procedure of effecting staged compression of the vapour stream and thus operating the intermediate evaporator and the bottoms evaporator with the vapours compressed to different extents surprisingly achieves energy savings.

1-12. (canceled)

13. A process for producing at least one alkali metal alkoxide of formula M_{A}OR, wherein R is methyl, and wherein M_{A} is a metal selected from sodium and potassium, wherein:
(α1) a reactant stream S_{AE1} comprising ROH is reacted with a reactant stream S_{AE2} comprising M_{A}OH in countercurrent in a reactive rectification column RR_{A} to produce a crude product RP_{A} comprising M_{A}OR, water, ROH, and M_{A}OH;
wherein a bottoms product stream S_{AP} comprising ROH and M_{A}OR is withdrawn at the lower end of RR_{A} and a vapour stream S_{AB} comprising water and ROH is withdrawn at the upper end of RR_{A};
(β) at least a portion of the vapour stream S_{AB} is employed as mixture G in step (a) of a process for workup of a mixture G comprising water and alcohol ROH;
wherein in the process for workup of a mixture G:
(a) the mixture G is passed into a rectification column RD_{A} and in RD_{A} is separated into at least one vapour stream S_{OA} comprising ROH which is withdrawn at the upper end of RD_{A} and at least one stream S_{UA} comprising water which is withdrawn at the lower end of RD_{A};
(b) at least one side stream S_{ZA} is withdrawn from RD_{A} and recycled to RD_{A};
(c) at least a portion of S_{OA} is compressed to afford a vapour stream S_{OA1} which is compressed relative to S_{OA};
(d) energy is transferred from a first portion S_{OA11} of the compressed vapour stream S_{OA1} to S_{ZA} before S_{ZA} is recycled to RD_{A};
(e) a portion S_{OA12} of the compressed vapour stream S_{OA1} that is distinct from S_{OA11} is subjected to further compression to produce a vapour stream S_{OA2} that is compressed relative to S_{OA11};
(f) energy is transferred from at least a portion of S_{OA2} to at least a portion S_{UA1} of S_{UA} before S_{UA1} is recycled to RD_{A}.

14. The process of claim 13, wherein in step (d), energy is transferred from S_{OA11} to S_{ZA} in an intermediate evaporator V_{ZRD}.

15. The process of claim 13, wherein, in step (f), energy is transferred from at least a portion of S_{OA2} to the at least a portion S_{UA1} of S_{UA} in a bottoms evaporator V_{SRD}.

16. The process of claim 13, wherein, once energy has been transferred from S_{OA11} to S_{ZA} according to step (d), energy is transferred from S_{OA11} to S_{OA} and/or once energy has been transferred from at least a portion of S_{OA2} to the at least a portion S_{UA1} of S_{UA} according to step (f), energy is transferred from at least a portion of S_{OA2} to S_{OA}.

17. The process of claim 13, wherein rectification column RD_{A} and reaction column RR_{A} are accommodated in one column shell, wherein the columns are at least partially separated from one another by a dividing wall extending to the bottom of the column.

18. The process of claim 13, wherein a portion of S_{OA} is employed as reactant stream S_{AE1} in step (α1).

19. The process of claim 13, wherein energy is transferred from at least a portion of a stream selected from S_{OA1}, and S_{OA2} to the crude product RP_{A}.

20. The process of claim 13, wherein, in a reactive rectification column RR_{C}, a reactant stream S_{CE1} comprising M_{c}OR′ is reacted in countercurrent with a reactant stream S_{CE2} comprising R″OH to produce a crude product RP_{C} comprising M_{c}OR″ and R′OH;
wherein a bottoms product stream S_{CP} comprising M_{C}OR″ is withdrawn at the lower end of RR_{C} and a vapour stream S_{CB} comprising R′OH is withdrawn at the upper end of RR_{C};
and wherein R′ and R″ are two distinct C₁ to C₆ hydrocarbon radicals and M_{C} is a metal selected from sodium and potassium;
and wherein energy is transferred from at least a portion of a stream selected from S_{OA1}, S_{OA2} to the crude product RP_{C}.

21. A process for producing at least one alkali metal alkoxide of formula M_{A}OR, wherein R is methyl, and wherein M_{A} is a metal selected from sodium, potassium, wherein:
(α1) a reactant stream S_{AE1} comprising ROH is reacted with a reactant stream S_{AE2} comprising M_{A}OH in countercurrent in a reactive rectification column RR_{A} to produce a crude product RP_{A} comprising M_{A}OR, water, ROH, M_{A}OH;
wherein a bottoms product stream S_{AP} comprising ROH and M_{A}OR is withdrawn at the lower end of RR_{A} and a vapour stream S_{AB} comprising water and ROH is withdrawn at the upper end of RR_{A};
(α2) simultaneously with and spatially separate from step (α1), a reactant stream S_{BE1} comprising ROH is reacted with a reactant stream S_{BE2} comprising M_{B}OH in countercurrent in a reactive rectification column RR_{B} to afford a crude product RP_{B} comprising M_{B}OR, water, ROH, M_{B}OH, wherein M_{B} is a metal selected from sodium and potassium;
wherein a bottoms product stream S_{BP} comprising ROH and M_{B}OR is withdrawn at the lower end of RR_{B} and a vapour stream S_{BB} comprising water and ROH is withdrawn at the upper end of RR_{B};
(β) at least a portion of the vapour stream S_{AB}, and at least a portion of the vapour stream S_{BB}, in admixture with S_{AB} or separate from S_{AB}, is employed as mixture G in step (a) of a process for workup of a mixture G comprising water and alcohol ROH;
wherein in the process for workup of a mixture G:
(a) the mixture G is passed into a rectification column RD_{A} and in RD_{A} is separated into at least one vapour stream S_{OA} comprising ROH which is withdrawn at the upper end of RD_{A} and at least one stream S_{UA} comprising water which is withdrawn at the lower end of RD_{A};
(b) at least one side stream S_{ZA} is withdrawn from RD_{A} and recycled to RD_{A};
(c) at least a portion of S_{OA} is compressed to afford a vapour stream S_{OA1} which is compressed relative to S_{OA};
(d) energy is transferred from a first portion S_{OA11} of the compressed vapour stream S_{OA1} to S_{ZA} before S_{ZA} is recycled to RD_{A};
(e) a portion S_{OA12} of the compressed vapour stream S_{OA1} that is distinct from S_{OA11} is subjected to further compression to produce a vapour stream S_{OA2} that is compressed relative to S_{OA11};
(f) energy is transferred from at least a portion of S_{OA2} to at least a portion S_{UA1} of S_{UA} before S_{UA1} is recycled to RD_{A}.

22. The process of claim 21, wherein in step (d), energy is transferred from S_{OA11} to S_{ZA} in an intermediate evaporator V_{ZRD}.

23. The process of claim 21, wherein in step (f), energy is transferred from at least a portion of S_{OA2} to the at least a portion S_{UA1} of S_{UA} in a bottoms evaporator V_{SRD}.

24. The process of claim 21, wherein once energy has been transferred from S_{OA11} to S_{ZA} according to step (d), energy is transferred from S_{OA11} to S_{OA} and/or once energy has been transferred from at least a portion of S_{OA2} to the at least a portion S_{UA1} of S_{UA} according to step (f) energy is transferred from at least a portion of S_{OA2} to S_{OA}.

25. The process of claim 21, wherein at least two of the columns selected from rectification column RD_{A}, reaction column RR_{A} and reaction column RR_{B} are accommodated in one column shell, wherein the columns are at least partially separated from one another by a dividing wall extending to the bottom of the column.

26. The process of claim 21, wherein a portion of S_{OA} is employed as reactant stream S_{AE1} in step (α1) and alternatively or in addition as reactant stream S_{BE1} in step (α2).

27. The process of claim 21, wherein energy is transferred from at least a portion of a stream selected from S_{OA1}, and S_{OA2} to the crude product RP_{A} and alternatively or in addition to the crude product RP_{B}.

28. The process of claim 21, wherein in a reactive rectification column RR_{C} a reactant stream S_{CE1} comprising M_{c}OR′ is reacted in countercurrent with a reactant stream S_{CE2} comprising R″OH to produce a crude product RP_{C} comprising M_{C}OR″ and R′OH;
wherein a bottoms product stream S_{CP} comprising M_{c}OR″ is withdrawn at the lower end of RR_{C} and a vapour stream S_{CB} comprising R′OH is withdrawn at the upper end of RR_{C};
and wherein R′ and R″ are two distinct C₁ to C₆ hydrocarbon radicals and M_{C} is a metal selected from sodium and potassium;
and wherein energy is transferred from at least a portion of a stream selected from S_{OA1}, S_{OA2} to the crude product RP_{C}.

29. The process of claim 28, wherein R′=methyl.

30. The process of claim 29, wherein the process produces S_{AP}, wherein R=methyl and wherein at least a portion of S_{AP} is employed as S_{CE1}.

31. The process of claim 29, wherein the process produces S_{BP}, wherein R=methyl and wherein at least a portion of S_{BP} is employed as S_{CE1}.

32. The process of claim 29, wherein R″=ethyl.
